SEQUENCE LISTING (35 sequences)

SEQ ID NO: 1 - RNA, 21 nt, Artificial Sequence
  siRNA
gggagucggugaaguacuuuu

SEQ ID NO: 2 - RNA, 21 nt, Artificial Sequence
  siRNA
ggaucggccagcugaauuauu

SEQ ID NO: 3 - RNA, 21 nt, Artificial Sequence
  siRNA
ggaaagccaccaagggaauuu

SEQ ID NO: 4 - RNA, 21 nt, Artificial Sequence
  siRNA
gagauaagcuuggcauuccuu

SEQ ID NO: 5 - RNA, 21 nt, Artificial Sequence
  siRNA
gaacguggcuuaugaguacuu

SEQ ID NO: 6 - RNA, 21 nt, Artificial Sequence
  siRNA
gcagguggauuacuauaaauu

SEQ ID NO: 7 - RNA, 21 nt, Artificial Sequence
  siRNA
cgaaccaucuuacugaauauu

SEQ ID NO: 8 - RNA, 21 nt, Artificial Sequence
  siRNA
caauugagcaguucaguuauu

SEQ ID NO: 9 - DNA, 4398 bp, Homo sapiens
gggcgacgagtgcgggcctcggagcgactgcagcggcggcggcggacgcggcctgaggcgagcggcggggcgtggggcggtgcctcggcccgggctcgccctcgccggcgggagcgtccatggcccccgggcgccggcggggcgcggccgcggcctgaggggccatgtccggggtggtgcggaccctcagccgctgcctgctgccggccgaggccggcggggcccgcgagcgcagggcgggcagcggcgcgcgcgacgcggagcgcgaggcccggaggcgtagccgcgacatcgacgcgctgctggcccgcgagcggcgcgcggtccggcgcctggtgaagatcctgctgctgggcgcgggcgagagcggcaagtccacgttcctcaagcagatgcgcatcatccacggccgcgagttcgaccagaaggcgctgctggagttccgcgacaccatcttcgacaacatcctcaagggctcaagggttcttgttgatgcacgagataagcttggcattccttggcagtattctgaaaatgagaagcatgggatgttcctgatggccttcgagaacaaggcggggctgcctgtggagccggccaccttccagctgtacgtcccggccctgagcgcactctggagggattctggcatcagggaggctttcagccggagaagcgagtttcagctgggggagtcggtgaagtacttcctggacaacttggaccggatcggccagctgaattactttcctagtaagcaagatatcctgctggctaggaaagccaccaagggaattgtggagcatgacttcgttattaagaagatcccctttaagatggtggatgtgggcggccagcggtcccagcgccagaagtggttccagtgcttcgacgggatcacgtccatcctgttcatggtctcctccagcgagtacgaccaggtcctcatggaggacaggcgcaccaaccggctggtggagtccatgaacatcttcgagaccatcgtcaacaacaagctcttcttcaacgtctccatcattctcttcctcaacaagatggacctcctggtggagaaggtgaagaccgtgagcatcaagaagcacttcccggacttcaggggcgacccgcacaggctggaggacgtccagcgctacctggtccagtgcttcgacaggaagagacggaaccgcagcaagccactcttccaccacttcaccaccgccatcgacaccgagaacgtccgcttcgtgttccatgctgtgaaagacaccatcctgcaggagaacctgaaggacatcatgctgcagtgagcgaggaagccccggggtttgtcgtcgttgagcagcccccacggctgtcggtcagactcttgggtgtgtgttgtctgtgtggtccttgagtgggtttctcggatccgtgccctggaatacctggctcaggaatgctgtcagaccagccagccagcgagctctaggcaaaaggacatggaaactgtcacgttagctactgaatcctgggggcgagtgaaactactgaaaatccgagtgatgatgttgtgaatacggaacacctaatcacacagcttgctttgcttttacagaaacgttcctctttttctgacgcagtttaattgaggaccgtgttgtgtgtgtatgtgtgtacacacgctctgtctttaatgacagaaacacaaaaaccagctggccttgcagacggcttttctaactcacaagtcttccctgagacagactaacctgaaagctttgcctaacagtagcttgtagagatccagtgcacgccgatgctgctaaactcagtgcctgagcccggccctgcagccccagccgcagtgtctgaaggccacctcccaaagggagcacgttgccttttcaaactcccgtgccgatttcctaagagcccctagtccaagcctctcagatgaagctgaggagccgtgcctaggatcccttcccagctctgaggacgggctgcagagctctgcaggtgtggattcaccttacgcccctacagcaggctcagcccttcccaccctgccccatgcccagcagcacaacacggagtgagacaggatgcccacggtgactgccgctccgtccgtgcacacacagcggtgctcttctccccttagccacccactgcccaacccaacggcaaagacacagaaaccaggtccccttgcagacggctctcccatcttcctgcaagtcatctgctcacacacagttggcagcacatagcgtttccttctttcagaaacattcctcttctggggcttcagaaagctggcaaggccactagcagagcttttgttaatgccccagctgcttggcgagctaacagctgacctttcgggaagcccacagacgctggaggaatcttgagtttctccaaactgccgctccaccagtgcctttggacagccgtgcctgttcgccgctctccctaagtctgattctcatcgaggcccctcgcttctatgactgtgcttgcagaagagtaaacactctcggatgccgctgtcctgggggagcccgcgggagcctgtgaatgttgatacgagctggccagtcctgggcccagctcacttgtccagctacctgccaggtggctttcactgtgtttaaaatacattgcattccaagctggtcccctctgtgtatcactctactgagaaatcctgcctagtgtgttttgggatgtgtcctagcatttacaagaaaatgaaaagcgtcctcttaattggcacccgaatgttgctgtggctcagtcacatatcccagggccctcgtcccgaggccgtgctgccccgagccccgagcccctctgcagctcacccttggcttgttttccgcaaacccggtaaacgcaagcccttggggcagatgcagaagcagaagagggaggggaaacctgcctctgggtcaccctgttagcacagcgttctcatcgggagacagcatggaactctctctcgcagtgctcgaggctgtgtgtcagtgtttgctgggcttgtggctccttttttggctggataaagaagtcgctgtttttgtactgcttctgtggctcttcacagacctcacggatgtgaccggagatgagtgccgatgaccacgttttaaaggagaaagagagctcctggtggggccctcggggtggtctcaggtcccatttgcagtctgcaacagtgacgcgcagcccggtccggagcgtggtgagctttgtttgccttctgggtcagctttcgctgtgtctcctgtgtgtgttagaatccagagcccagaggaagtgcaagcgggtcctccgccaacggggagagcctcttcgcggcgctgttggcgacagcagcgctgtgattcgcgtagcaggggagttgtttgaaacaccttcctgagtagtccggccttgtcaatgagtgcttgttttcctttaaacagtctgacatatttactcgtcactttcaaaccagaagcatgagaggaaggagatattgtggggtccgtttaactcgatagaaagcgcagggggatggcccccggcgcgggctcttgacccgctcagcgctgaccccaccgccctggccgaggcacttggccttgctgagctggacttcctcctcctcctcctcatgaccggggtgaattagaacgtttttaaagacacccccttccaaattctgtaacacattgtaattggagaagaaggaaactctgcaaggctaaactgtcattcacaacttggctacacatagactctagtcagttttgtctccagaaccttaggcttttgtattttttaattttaatttcactgttaatccttattgtcttttttattaagatgttggaaaagcaggaggtagttgtgcctcaattattgcaaaaatgtaacaataaagttcctcaaaataagatctgttcctcatagctatactgtgtacacataagacgcatatagggttttactgaaatctatttttaactcttatgttcgtagagaaattgtttcaaggattttgagtcataggtctgtaatttatagagatctctagaattcttattgtaattttcctacttctttgataaaagaaaaataagtcagattgttaactccaagattgaaaaaaaaaactcttgaaagaagattattagttgtaactaatttaggggttctgggcacagacatctaacctggtattgtaaggcagaggctcccattggaatggtagtggtccgggtcagttgttcatggtgtaagctttgcacagtgtattaacattgggagggtctggcttgaaaatttggccaccctcagcctctgaatgtttattaaaataaatttagtctttctttgcttaatataaaaaaaaaaaaaaaa

SEQ ID NO: 10 - protein, 381 aa, Homo sapiens
MSGVVRTLSRCLLPAEAGGARERRAGSGARDAEREARRRSRDIDALLARERRAVRRLVKILLLGAGESGKSTFLKQMRIIHGREFDQKALLEFRDTIFDNILKGSRVLVDARDKLGIPWQYSENEKHGMFLMAFENKAGLPVEPATFQLYVPALSALWRDSGIREAFSRRSEFQLGESVKYFLDNLDRIGQLNYFPSKQDILLARKATKGIVEHDFVIKKIPFKMVDVGGQRSQRQKWFQCFDGITSILFMVSSSEYDQVLMEDRRTNRLVESMNIFETIVNNKLFFNVSIILFLNKMDLLVEKVKTVSIKKHFPDFRGDPHRLEDVQRYLVQCFDRKRRNRSKPLFHHFTTAIDTENVRFVFHAVKDTILQENLKDIMLQ

SEQ ID NO: 11 - DNA, 7233 bp, Homo sapiens
gacggcacggggcggggcctcggggaccccggcaagcccgcgcacttggcaggagctgtagctaccgccgtccgcgcctccaaggtttcacggcttcctcagcagagactcgggctcgtccgccatgtccgccgcagacgaggttgacgggctgggcgtggcccggccgcactatggctctgtcctggataatgaaagacttactgcagaggagatggatgaaaggagacgtcagaacgtggcttatgagtacctttgtcatttggaagaagcgaagaggtggatggaagcatgcctaggggaagatctgcctcccaccacagaactggaggaggggcttaggaatggggtctaccttgccaaactggggaacttcttctctcccaaagtagtgtccctgaaaaaaatctatgatcgagaacagaccagatacaaggcgactggcctccactttagacacactgataatgtgattcagtggttgaatgccatggatgagattggattgcctaagattttttacccagaaactacagatatctatgatcgaaagaacatgccaagatgtatctactgtatccatgcactcagtttgtacctgttcaagctaggcctggcccctcagattcaagacctatatggaaaggttgacttcacagaagaagaaatcaacaacatgaagactgagttggagaagtatggcatccagatgcctgcctttagcaagattgggggcatcttggctaatgaactgtcagtggatgaagccgcattacatgctgctgttattgctattaatgaagctattgaccgtagaattccagccgacacatttgcagctttgaaaaatccgaatgccatgcttgtaaatcttgaagagcccttggcatccacttaccaggatatactttaccaggctaagcaggacaaaatgacaaatgctaaaaacaggacagaaaactcagagagagaaagagatgtttatgaggagctgctcacgcaagctgaaattcaaggcaatataaacaaagtcaatacattttctgcattagcaaatatcgacctggctttagaacaaggagatgcactggccttgttcagggctctgcagtcaccagccctggggcttcgaggactgcagcaacagaatagcgactggtacttgaagcagctcctgagtgataaacagcagaagagacagagtggtcagactgaccccctgcagaaggaggagctgcagtctggagtggatgctgcaaacagtgctgcccagcaatatcagagaagattggcagcagtagcactgattaatgctgcaatccagaagggtgttgctgagaagactgttttggaactgatgaatcccgaagcccagctgccccaggtgtatccatttgccgccgatctctatcagaaggagctggctaccctgcagcgacaaagtcctgaacataatctcacccacccagagctctctgtcgcagtggagatgttgtcatcggtggccctgatcaacagggcattggaatcaggagatgtgaatacagtgtggaagcaattgagcagttcagttactggtcttaccaatattgaggaagaaaactgtcagaggtatctcgatgagttgatgaaactgaaggctcaggcacatgcagagaataatgaattcattacatggaatgatatccaagcttgcgtggaccatgtgaacctggtggtgcaagaggaacatgagaggattttagccattggtttaattaatgaagccctggatgaaggtgatgcccaaaagactctgcaggccctacagattcctgcagctaaacttgagggagtccttgcagaagtggcccagcattaccaagacacgctgattagagcgaagagagagaaagcccaggaaatccaggatgagtcagctgtgttatggttggatgaaattcaaggtggaatctggcagtccaacaaagacacccaagaagcacagaagtttgccttaggaatctttgccattaatgaggcagtagaaagtggtgatgttggcaaaacactgagtgcccttcgctcccctgatgttggcttgtatggagtcatccctgagtgtggtgaaacttaccacagtgatcttgctgaagccaagaagaaaaaactggcagtaggagataataacagcaagtgggtgaagcactgggtaaaaggtggatattattattaccacaatctggagacccaggaaggaggatgggatgaacctccaaattttgtgcaaaattctatgcagctttctcgggaggagatccagagttctatctctggggtgactgccgcatataaccgagaacagctgtggctggccaatgaaggcctgatcaccaggctgcaggctcgctgccgtggatacttagttcgacaggaattccgatccaggatgaatttcctgaagaaacaaatccctgccatcacctgcattcagtcacagtggagaggatacaagcagaagaaggcatatcaagatcggttagcttacctgcgctcccacaaagatgaagttgtaaagattcagtccctggcaaggatgcaccaagctcgaaagcgctatcgagatcgcctgcagtacttccgggaccatataaatgacattatcaaaatccaggcttttattcgggcaaacaaagctcgggatgactacaagactctcatcaatgctgaggatcctcctatggttgtggtccgaaaatttgtccacctgctggaccaaagtgaccaggattttcaggaggagcttgaccttatgaagatgcgggaagaggttatcaccctcattcgttctaaccagcagctggagaatgacctcaatctcatggatatcaaaattggactgctagtgaaaaataagattacgttgcaggatgtggtttcccacagtaaaaaacttaccaaaaaaaataaggaacagttgtctgatatgatgatgataaataaacagaagggaggtctcaaggctttgagcaaggagaagagagagaagttggaagcttaccagcacctgttttatttattgcaaaccaatcccacctatctggccaagctcatttttcagatgccccagaacaagtccaccaagttcatggactctgtaatcttcacactctacaactacgcgtccaaccagcgagaggagtacctgctcctgcggctctttaagacagcactccaagaggaaatcaagtcgaaggtagatcagattcaagagattgtgacaggaaatcctacggttattaaaatggttgtaagtttcaaccgtggtgcccgtggccagaatgccctgagacagatcttggccccagtcgtgaaggaaattatggatgacaaatctctcaacatcaaaactgaccctgtggatatttacaaatcttgggttaatcagatggagtctcagacaggagaggcaagcaaactgccctatgatgtgacccctgagcaggcgctagctcatgaagaagtgaagacacggctagacagctccatcaggaacatgcgggctgtgacagacaagtttctctcagccattgtcagctctgtggacaaaatcccttatgggatgcgcttcattgccaaagtgctgaaggactcgttgcatgagaagttccctgatgctggtgaggatgagctgctgaagattattggtaacttgctttattatcgatacatgaatccagccattgttgctcctgatgcctttgacatcattgacctgtcagcaggaggccagcttaccacagaccaacgccgaaatctgggctccattgcaaaaatgcttcagcatgctgcttccaataagatgtttctgggagataatgcccacttaagcatcattaatgaatatctttcccagtcctaccagaaattcagacggtttttccaaactgcttgtgatgtcccagagcttcaggataaatttaatgtggatgagtactctgatttagtaaccctcaccaaaccagtaatctacatttccattggtgaaatcatcaacacccacactctcctgttggatcaccaggatgccattgctccggagcacaatgatccaatccacgaactgctggacgacctcggcgaggtgcccaccatcgagtccctgataggggaaagctctggcaatttaaatgacccaaataaggaggcactggctaagacggaagtgtctctcaccctgaccaacaagttcgacgtgcctggagatgagaatgcagaaatggatgctcgaaccatcttactgaatacaaaacgtttaattgtggatgtcatccggttccagccaggagagaccttgactgaaatcctagaaacaccagccaccagtgaacaggaagcagaacatcagagagccatgcagagacgtgctatccgtgatgccaaaacacctgacaagatgaaaaagtcaaaatctgtaaaggaagacagcaacctcactcttcaagagaagaaagagaagatccagacaggtttaaagaagctaacagagcttggaaccgtggacccaaagaacaaataccaggaactgatcaacgacattgccagggatattcggaatcagcggaggtaccgacagaggagaaaggccgaactagtgaaactgcaacagacatacgctgctctgaactctaaggccaccttttatggggagcaggtggattactataaaagctatatcaaaacctgcttggataacttagccagcaagggcaaagtctccaaaaagcctagggaaatgaaaggaaagaaaagcaaaaagatttctctgaaatatacagcagcaagactacatgaaaaaggagttcttctggaaattgaggacctgcaagtgaatcagtttaaaaatgttatatttgaaatcagtccaacagaagaagttggagacttcgaagtgaaagccaaattcatgggagttcaaatggagacttttatgttacattatcaggacctgctgcagctacagtatgaaggagttgcagtcatgaaattatttgatagagctaaagtaaatgtcaacctcctgatcttccttctcaacaaaaagttctacgggaagtaattgatcgtttgctgccagcccagaaggatgaaggaaagaagcacctcacagctcctttctaggtccttctttcctcattggaagcaaagacctagccaacaacagcacctcaatctgatacactcccgatgccacatttttaactcctctcgctctgatgggacatttgttacccttttttcatagtgaaattgtgtttcaggcttagtctgacctttctggtttcttcattttcttccattacttaggaaagagtggaaactccactaaaatttctctgtgttgttacagtcttagaggttgcagtactatattgtaagctttggtgtttgtttaattagcaatagggatggtaggattcaaatgtgtgtcatttagaagtggaagctattagcaccaatgacataaatacatacaagacacacaactaaaatgtcatgttattaacagttattaggttgtcatttaaaaataaagttcctttatatttctgtcccatcaggaaaactgaaggatatggggaatcattggttatcttccattgtgtttttctttatggacaggagctaatggaagtgacagtcatgttcaaaggaagcatttctagaaaaaaggagataatgtttttaaatttcattatcaaacttgggcaattctgtttgtgtaactccccgactagtggatgggagagtcccattgctaaaattcagctactcagataaattcagaatgggtcaaggcacctgcctgtttttgttggtgcacagagattgacttgattcagagagacaattcactccatccctatggcagaggaatgggttagccctaatgtagaatgtcattgtttttaaaactgttttatatcttaagagtgccttattaaagtatagatgtatgtcttaaaatgtgggtgataggaattttaaagatttatataatgcatcaaaagccttagaataagaaaagctttttttaaattgctttatctgtatatctgaactcttgaaacttatagctaaaacactaggatttatctgcagtgttcagggagataattctgcctttaattgtctaaaacaaaaacaaaaccagccaacctatgttacacgtgagattaaaaccaattttttccccattttttctccttttttctcttgctgcccacattgtgcctttattttatgagccccagttttctgggcttagtttaaaaaaaaaatcaagtctaaacattgcatttagaaagcttttgttcttggataaaaagtcatacactttaaaaaaaaaaaaaactttttccaggaaaatatattgaaatcatgctgctgagcctctattttctttctttgatgttttgattcagtattcttttatcataaatttttagcatttaaaaattcactgatgtacattaagccaataaactgctttaatgaataacaaactatgtagtgtgtccctattataaatgcattggagaagtatttttatgagactctttactcaggtgcatggttacagcccacagggaggcatggagtgccatggaaggattcgccactacccagaccttgttttttgttgtattttggaagacaggttttttaaagaaacattttcctcagattaaaagatgatgctattacaactagcattgcctcaaaaactgggaccaaccaaagtgtgtcaaccctgtttccttaaaagaggctatgaatcccaaaggccacatccaagacaggcaataatgagcagagtttacagctcctttaataaaatgtgtcagtaattttaaggtttatagttccctcaacacaattgctaatgcagaatagtgtaaaatgcgcttcaagaatgttgatgatgatgatatagaattgtggctttagtagcacagaggatgccccaacaaactcatggcgttgaaaccacacagttctcattactgttatttattagctgtagcattctctgtctcctctctctcctcctttgaccttctcctcgaccagccatcatgacatttaccatgaatttacttcctcccaagagtttggactgcccgtcagattgttgctgcacatagttgcctttgtatctctgtatgaaataaaaggtcatttgttcatgtt

SEQ ID NO: 12 - protein, 1656 aa, Homo sapiens
MSAADEVDGLGVARPHYGSVLDNERLTAEEMDERRRQNVAYEYLCHLEEAKRWMEACLGEDLPPTTELEEGLRNGVYLAKLGNFFSPKVVSLKKIYDREQTRYKATGLHFRHTDNVIQWLNAMDEIGLPKIFYPETTDIYDRKNMPRCIYCIHALSLYLFKLGLAPQIQDLYGKVDFTEEEINNMKTELEKYGIQMPAFSKIGGILANELSVDEAALHAAVIAINEAIDRRIPADTFAALKNPNAMLVNLEEPLASTYQDILYQAKQDKMTNAKNRTENSERERDVYEELLTQAEIQGNINKVNTFSALANIDLALEQGDALALFRALQSPALGLRGLQQQNSDWYLKQLLSDKQQKRQSGQTDPLQKEELQSGVDAANSAAQQYQRRLAAVALINAAIQKGVAEKTVLELMNPEAQLPQVYPFAADLYQKELATLQRQSPEHNLTHPELSVAVEMLSSVALINRALESGDVNTVWKQLSSSVTGLTNIEEENCQRYLDELMKLKAQAHAENNEFITWNDIQACVDHVNLVVQEEHERILAIGLINEALDEGDAQKTLQALQIPAAKLEGVLAEVAQHYQDTLIRAKREKAQEIQDESAVLWLDEIQGGIWQSNKDTQEAQKFALGIFAINEAVESGDVGKTLSALRSPDVGLYGVIPECGETYHSDLAEAKKKKLAVGDNNSKWVKHWVKGGYYYYHNLETQEGGWDEPPNFVQNSMQLSREEIQSSISGVTAAYNREQLWLANEGLITRLQARCRGYLVRQEFRSRMNFLKKQIPAITCIQSQWRGYKQKKAYQDRLAYLRSHKDEVVKIQSLARMHQARKRYRDRLQYFRDHINDIIKIQAFIRANKARDDYKTLINAEDPPMVVVRKFVHLLDQSDQDFQEELDLMKMREEVITLIRSNQQLENDLNLMDIKIGLLVKNKITLQDVVSHSKKLTKKNKEQLSDMMMINKQKGGLKALSKEKREKLEAYQHLFYLLQTNPTYLAKLIFQMPQNKSTKFMDSVIFTLYNYASNQREEYLLLRLFKTALQEEIKSKVDQIQEIVTGNPTVIKMVVSFNRGARGQNALRQILAPVVKEIMDDKSLNIKTDPVDIYKSWVNQMESQTGEASKLPYDVTPEQALAHEEVKTRLDSSIRNMRAVTDKFLSAIVSSVDKIPYGMRFIAKVLKDSLHEKFPDAGEDELLKIIGNLLYYRYMNPAIVAPDAFDIIDLSAGGQLTTDQRRNLGSIAKMLQHAASNKMFLGDNAHLSIINEYLSQSYQKFRRFFQTACDVPELQDKFNVDEYSDLVTLTKPVIYISIGEIINTHTLLLDHQDAIAPEHNDPIHELLDDLGEVPTIESLIGESSGNLNDPNKEALAKTEVSLTLTNKFDVPGDENAEMARTILLNTKRLIVDVIRFQPGETLTEILETPATSEQEAEHQRAMQRRAIRDAKTPDKMKKSKSVKEDSNLTLQEKKEKIQTGLKKLTELGTVDPKNKYQELINDIARDIRNQRRYRQRRKAELVKLQQTYAALNSKATFYGEQVDYYKSYIKTCLDNLASKGKVSKKPREMKGKKSKKISLKYTAARLHEKGVLLEIEDLQVNQFKNVIFEISPTEEVGDFEVKAKFMGVQMETFMLHYQDLLQLQYEGVAVMKLFDRAKVNVNLLIFLLNKKFYGK

SEQ ID NO: 13 - DNA, 21 bp, Homo sapiens
gcgacaccatcttcgacaaca

SEQ ID NO: 14 - DNA, 17 bp, Artificial Sequence
  PCR Primer
gtttgtcgtcgttgagc

SEQ ID NO: 15 - DNA, 17 bp, Artificial Sequence
  PCR Primer
agtagtttcactcgccc

SEQ ID NO: 16 - DNA, 20 bp, Artificial Sequence
  PCR Primer
cccaaagaacaaataccagg

SEQ ID NO: 17 - DNA, 19 bp, Artificial Sequence
  PCR Primer
ggctaagttatccaagcag

SEQ ID NO: 18 - DNA, 18 bp, Artificial Sequence
  PCR Primer
attagagagggtgctcag

SEQ ID NO: 19 - DNA, 17 bp, Artificial Sequence
  PCR Primer
atgaatggctttgctcc

SEQ ID NO: 20 - DNA, 19 bp, Artificial Sequence
  PCR Primer
agttacaccattctttgcc

SEQ ID NO: 21 - DNA, 16 bp, Artificial Sequence
  PCR Primer
gcaccttgggacttga

SEQ ID NO: 22 - DNA, 17 bp, Artificial Sequence
  PCR Primer
catcagcacaactacgc

SEQ ID NO: 23 - DNA, 16 bp, Artificial Sequence
  PCR Primer
ctcgttcctcctctgg

SEQ ID NO: 24 - DNA, 17 bp, Artificial Sequence
  PCR Primer
cttgaaggtgctcatct

SEQ ID NO: 25 - DNA, 17 bp, Artificial Sequence
  PCR Primer
ggaccctactcctccag

SEQ ID NO: 26 - DNA, 20 bp, Artificial Sequence
  PCR Primer
gaagacctaaatgacctcag

SEQ ID NO: 27 - DNA, 17 bp, Artificial Sequence
  PCR Primer
ttttcgcttggcacaat

SEQ ID NO: 28 - DNA, 20 bp, Artificial Sequence
  PCR Primer
tgtaaggggatggactattt

SEQ ID NO: 29 - DNA, 18 bp, Artificial Sequence
  PCR Primer
aacattctggagcatacc

SEQ ID NO: 30 - DNA, 19 bp, Artificial Sequence
  PCR Primer
agagacaggttacattccc

SEQ ID NO: 31 - DNA, 20 bp, Artificial Sequence
  PCR Primer
tcccaatcacggatagaaag

SEQ ID NO: 32 - DNA, 17 bp, Artificial Sequence
  PCR Primer
tcgttagtccacggtct

SEQ ID NO: 33 - DNA, 16 bp, Artificial Sequence
  PCR Primer
aactggtccttgctga

SEQ ID NO: 34 - DNA, 20 bp, Artificial Sequence
  PCR Primer
agcattcagtagagaaagtc

SEQ ID NO: 35 - DNA, 18 bp, Artificial Sequence
  PCR Primer
gtcttccagtaagtcagg

(19) United States
(12) Patent Application Publication
(10) Pub. No.: US 2010/0003257 A1
(43) Pub. Date: Jan. 7, 2010

(54) Diagnosis of nasopharyngeal carcinoma and suppression of nasopharyngeal carcinoma invasion

(75) Inventors: Jyh-Lyh Juang, Zhunan Town (TW); Shu-Chen Liu, Sijhih CIty (TW)

Correspondence Address: OCCHIUTI ROHLICEK & TSAO, LLP, 10 FAWCETT STREET, CAMBRIDGE, MA, 02138, US

(73) Assignee: National Health Research Institutes, Zhunan Town (TW)

(21) Appl. No.: 12/455,033

(22) Filed: May 27, 2009

Related U.S. Application Data
(60) Provisional application No. 61/128,940, filed on May 27, 2008.

(51) Int. Cl. A61K 39/395; C12Q 1/68; C12Q 1/02; A61K 31/7105

(52) U.S. Cl. 424/139.1; 435/6; 435/29; 514/44 A

(57) ABSTRACT

A diagnostic method for NPC based on the activation level of the Gα₁₂ signaling pathway in a subject. Also disclosed is a method of inhibiting NPC invasion by suppressing the Gα₁₂ signaling pathway or reducing the level of IQ motif containing GTPase protein 1 in a NPC patient.

### RELATED APPLICATION

This application claims priority to U.S. Provisional Application No. 61/128,940, filed on May 27, 2008, the content of which is hereby incorporated by reference in its entirety.

### COMPUTER-READABLE APPENDICES

Tables of gene expression data referred to in the specification are provided in Appendices I, II, and III, all of which are in computer-readable form.

### BACKGROUND OF THE INVENTION

Nasopharyngeal carcinoma (NPC) is a head-and-neck cancer originating from the mucosal epithelium of the nasopharynx. While very rare in western countries, NPC is common in certain regions of East Asia and Africa. Multiple factors have been implicated in its causation, including Epstein-Barr viral infection, genetic background, environmental factors, and diet habit.

NPC is highly invasive and metastatic, resulting in a high mortality rate. Early diagnosis and suppression of cancer cell invasion would be effective approaches in treating NPC.

### SUMMARY OF THE INVENTION

This invention is based on the unexpected discoveries that (1) certain genes involved in the guanine nucleotide-binding protein alpha-12 (Gα₁₂) signaling pathway are significantly over-expressed in NPC tumor samples, and (2) inhibiting the Gα₁₂ signaling pathway or suppressing the expression level of IQ motif-containing GTPase activating protein 1 (IQGAP1) reduces NPC tumor cell mobility, a mechanism underlying tumor cell invasion.

Accordingly, one aspect of this invention features a method for diagnosing NPC by determining in a nasal sample obtained from a test subject an expression level of a gene involved in the Gα₁₂ signaling pathway (e.g., genes of Gα₁₂, Rho guanine nucleotide exchange factor 12, RhoA, SLC9A1, Rho-associated coiled-coil containing protein kinase, profiling 1, and JNK). If the expression level (i.e., the protein level or the mRNA level) of the gene in that nasal sample is either elevated or reduced relative to that in a nasal sample obtained from a healthy subject, it indicates that the test subject has NPC.

In another aspect, this invention provides a method of inhibiting NPC invasion by administering to a subject suffering from NPC an effective amount of an agent that suppresses the Gα₁₂ signaling pathway. The term “NPC invasion” used herein refers to a process in which cancer cells break away from its initiation site and crawl through the surrounding tissues to move into the bloodstream or the lymphatic system, and subsequently spread through the body to establish a secondary tumor at another site. In one example, the agent useful for inhibiting NPC invasion is a small molecule (e.g., Y-27632 and dimethyl BAPTA) or an antibody that binds to and inhibits the activity of a protein involved in the Gα₁₂ signaling pathway. In another example, the agent is one or more compounds (e.g., small interfering RNAs) that inhibit expression of a gene involved in the Gα₁₂ signaling pathway. Examples of small interfering RNAs (siRNAs) that inhibit Gα₁₂ gene expression include, but are not limited to, siRNAs each containing the nucleotide sequence of 5′-GGGAGUCGGUGAAGUACUUUU-3′, 5′-GGAUCGGCCAGCUGAAUUAUU-3′,5′-GGAAAGCCACCAAGGGAAUUU-3′, or 5′-GAGAUAAGCUUGGCAUUCCUU-3′.

In yet another aspect, the present invention provides a method of inhibiting NPC invasion by administering to a subject in need thereof an effective amount of an agent that reduces the level of IQ motif containing GTPase activating protein 1 (IQGAP1). This agent can be an antibody that specifically binds to IQGAP1 or an interfering RNA that suppresses expression of IQGAP1, e.g., small interfering RNAs each having the nucleotide sequence of 5′-GAACGUGGCUUAUGAGUACUU-3′,5′-GCAGGUGGAUUACUAUAAAUU-3′, 5′-CGAACCAUCUUACUGAAUAUU-3′, or 5′-CAAUUGAGCAGUUCAGUUAUU-3′.

Also within the scope of this invention is a method for screening a compound that suppresses NPC invasion. This method includes at least the following steps: (a) contacting a candidate compound with a NPC cell, (b) examining an activation level of the Gα₁₂ signaling pathway in the presence of the candidate compound and an activation level of the Gα₁₂ signaling pathway in the absence of the candidate compound, and (c) determining whether the candidate compound is capable of suppressing NPC invasion—if the activation level of the Gα₁₂ signaling pathway in the presence of the candidate compound is lower than that in the absence of the candidate compound, then the candidate compound possesses the activity of suppressing NPC invasion. The activation level of the Gα₁₂ signaling pathway can be indicated by the expression level of a gene involved in the Gα₁₂ signaling pathway (e.g., Gα₁₂), by cell morphology, or by the expression level of a gene downstream of the Gα₁₂ signaling pathway (e.g., the IQGAP1 gene).

The details of one or more embodiments of the invention are set forth in the description below. Other features or advantages of the present invention will be apparent from the following drawings, detailed description of several embodiments, and also from the appending claims.

### BRIEF DESCRIPTION OF THE DRAWINGS

The drawings are first described.

FIG. 1 is a diagram showing the Gα_{12/13} signaling pathway and the major components thereof.

FIG. 2 is a chart showing the expression levels of Gα₁₂ in primary nasopharyngeal epithelium (NPE) cells, primary nasopharyngeal carcinoma (NPC) cells, and NPC cell lines.

FIG. 3 is a diagram showing the effect of inhibiting Gα₁₂ expression via RNA interference on NPC cell mobility. A: a chart showing Gα₁₂ mRNA levels in two NPC cell lines, i.e., CNE1 and NPC-TW06, in the presence of Gα₁₂ siRNAs or a control siRNA via QRT-PCR analysis. B: a photo showing wound healing effects in the presence of Gα₁₂ siRNAs or a control siRNA. C: a photo showing invasion of NPC cells transfected with a control siRNA or Gα₁₂ siRNAs via Marigel invasion assays. D: a chart showing percentages of invaded cells.

FIG. 4 is a diagram showing the effect of inhibiting Gα₁₂ expression via RNA interference on NPC cell proliferation.

### DETAILED DESCRIPTION OF THE INVENTION

In one aspect, this invention provides a diagnostic method for NPC in a subject who is suspected of having have NPC based on the expression level of one or more genes that are differentially expressed in NPC. The subject can be one who is suffering from one or more symptoms associated with NPC, or one who has a family history of NPC. A gene differentially expressed in NPC has an elevated or reduced expression level in the nasopharynx- and its surrounding tissues of a NPC patient relative to that in the nasopharynx and its surrounding tissues of a healthy subject. Such genes can be identified by comparing gene expression profiles of NPC patients and healthy subjects via, e.g., microarray assays. See Examples 1 and 3 below. As an example, Appendix I includes gene expression data obtained from NPC patients and healthy subjects. Conventional statistical analysis has revealed a number of genes that are differentially expressed in NPC. These genes include those involved in RNA processing, transcription, chromatin architecture, protein modification, macromolecule (e.g., DNA and RNA) metabolism, organelle organization and biogenesis, ion transport, neuropeptide signaling pathway, and ubiquitin cycle. See Appendix III.

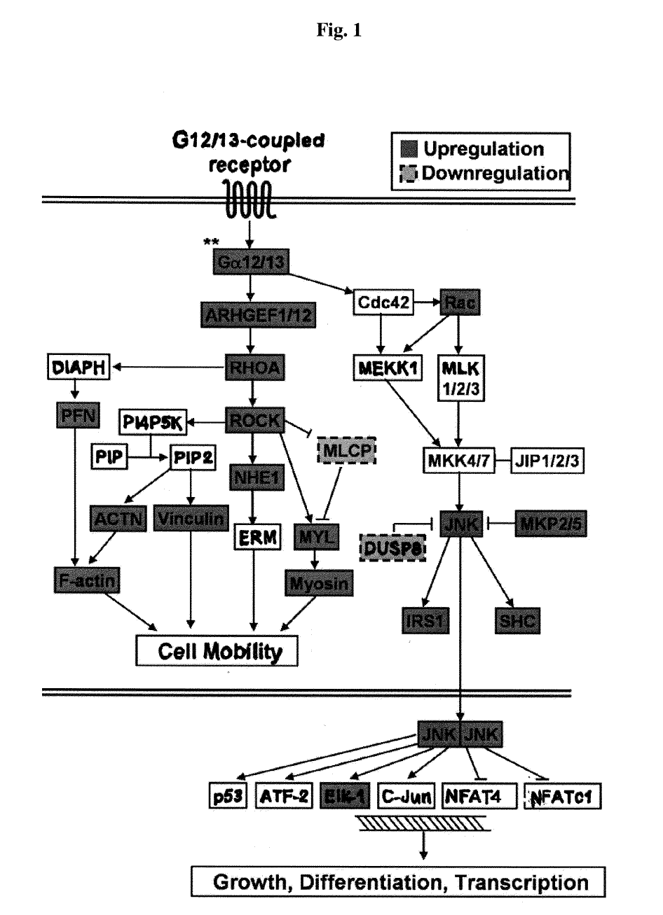

In a preferred embodiment, one or more genes involved in the Gα₁₂ signaling pathway (illustrated in FIG. 1) are used in this diagnostic method. A gene involved in this signaling pathway refers to a gene, whose protein product is either up-regulated or down-regulated once the signaling pathway is activated. The major members involved in the Gα₁₂ signaling pathway are also illustrated in FIG. 1. Alternatively, the expression level of a gene regulated by the Gα₁₂ signaling pathway, e.g., IQGAP1, can be used as a marker for detecting NPC invasion.

In the diagnostic method of this invention, the expression level of any of the genes mentioned above can be determined either at the mRNA level or at the protein level. Methods for quantification of mRNAs or proteins are well known in the art, e.g., real-time PCR and immunohistochemical staining. If the mRNA or protein of a gene being tested is either elevated or reduced in a patient relative to that in a healthy human subject, it indicates that the subject has NPC.

In another aspect, the present invention features a method of inhibiting NPC invasion in a subject who suffers from NPC by administering to the subject an effective amount of an agent that suppresses the Gα₁₂ signaling pathway or reduces the level of IQGAP1. The term “inhibiting invasion” refers to slowing and/or suppressing the spread of neoplastic cells to a site remote from the primary growth area, preferably by at least 10%, more preferably by at least 50%. This can be determined by the methods set forth in the Examples and other methods known in the art. “An effective amount” as used herein refers to the amount of each active agent which, upon administration with one or more other active agents to a subject in need thereof, is required to confer therapeutic effect on the subject. Effective amounts vary, as recognized by those skilled in the art, depending on route of administration, excipient usage, and the co-usage with other active agents. This method can be performed alone or in conjunction with other drugs or therapy.

The agent used in the just-described method can be one or more compound that inhibits expression of a gene involved in the Gα₁₂ signaling pathway, e.g., the Gα₁₂ gene, or suppresses the expression of the IQGAP1 gene. The nucleotide sequences of these two genes and their encoded amino acid sequences are shown below:

```
Nucleotide sequence of human Gα₁₂
      1 gggcgacgag tgcgggcctc ggagcgactg cagcggcggc ggcggacgcg gcctgaggcg
    61 agcggcgggg cgtggggcgg tgcctcggcc cgggctcgcc ctcgccggcg ggagcgtcca
  121 tggcccccgg gcgccggcgg ggcgcggccg cggcctgagg ggccatgtcc ggggtggtgc
  181 ggaccctcag ccgctgcctg ctgccggccg aggccggcgg ggcccgcgag cgcagggcgg
  241 gcagcggcgc gcgcgacgcg gagcgcgagg cccggaggcg tagccgcgac atcgacgcgc
  301 tgctggcccg cgagcggcgc gcggtccggc gcctggtgaa gatcctgctg ctgggcgcgg
  361 gcgagagcgg caagtccacg ttcctcaagc agatgcgcat catccacggc cgcgagttcg
  421 accagaaggc gctgctggag ttccgcgaca ccatcttcga caacatcctc aagggctcaa
  481 gggttcttgt tgatgcacga gataagcttg gcattccttg gcagtattct gaaaatgaga
  541 agcatgggat gttcctgatg gccttcgaga acaaggcggg gctgcctgtg gagccggcca
  601 ccttccagct gtacgtcccg gccctgagcg cactctggag ggattctggc atcagggagg
  661 ctttcagccg gagaagcgag tttcagctgg gggagtcggt gaagtacttc ctggacaact
  721 tggaccggat cggccagctg aattactttc ctagtaagca agatatcctg ctggctagga
  781 aagccaccaa gggaattgtg gagcatgact tcgttattaa gaagatcccc tttaagatgg
  841 tggatgtggg cggccagcgg tcccagcgcc agaagtggtt ccagtgcttc gacgggatca
  901 cgtccatcct gttcatggtc tcctccagcg agtacgacca ggtcctcatg gaggacaggc
  961 gcaccaaccg gctggtggag tccatgaaca tcttcgagac catcgtcaac aacaagctct
1021 tcttcaacgt ctccatcatt ctcttcctca acaagatgga cctcctggtg gagaaggtga
1081 agaccgtgag catcaagaag cacttcccgg acttcagggg cgacccgcac aggctggagg
1141 acgtccagcg ctacctggtc cagtgcttcg acaggaagag acggaaccgc agcaagccac
1201 tcttccacca cttcaccacc gccatcgaca ccgagaacgt ccgcttcgtg ttccatgctg
1261 tgaaagacac catcctgcag gagaacctga aggacatcat gctgcagtga gcgaggaagc
1321 cccggggttt gtcgtcgttg agcagccccc acggctgtcg gtcagactct tgggtgtgtg
1381 ttgtctgtgt ggtccttgag tgggtttctc ggatccgtgc cctggaatac ctggctcagg
1441 aatgctgtca gaccagccag ccagcgagct ctaggcaaaa ggacatggaa actgtcacgt
1501 tagctactga atcctggggg cgagtgaaac tactgaaaat ccgagtgatg atgttgtgaa
1561 tacggaacac ctaatcacac agcttgcttt gcttttacag aaacgttcct ctttttctga
1621 cgcagtttaa ttgaggaccg tgttgtgtgt gtatgtgtgt acacacgctc tgtctttaat
1681 gacagaaaca caaaaaccag ctggccttgc agacggcttt tctaactcac aagtcttccc
1741 tgagacagac taacctgaaa gctttgccta acagtagctt gtagagatcc agtgcacgcc
1801 gatgctgcta aactcagtgc ctgagcccgg ccctgcagcc ccagccgcag tgtctgaagg
1861 ccacctccca aagggagcac gttgcctttt caaactcccg tgccgatttc ctaagagccc
1921 ctagtccaag cctctcagat gaagctgagg agccgtgcct aggatccctt cccagctctg
1981 aggacgggct gcagagctct gcaggtgtgg attcacctta cgcccctaca gcaggctcag
2041 cccttcccac cctgccccat gcccagcagc acaacacgga gtgagacagg atgcccacgg
2101 tgactgccgc tccgtccgtg cacacacagc ggtgctcttc tccccttagc cacccactgc
2161 ccaacccaac ggcaaagaca cagaaaccag gtccccttgc agacggctct cccatcttcc
2221 tgcaagtcat ctgctcacac acagttggca gcacatagcg tttccttctt tcagaaacat
2281 tcctcttctg gggcttcaga aagctggcaa ggccactagc agagcttttg ttaatgcccc
2341 agctgcttgg cgagctaaca gctgaccttt cgggaagccc acagacgctg gaggaatctt
2401 gagtttctcc aaactgccgc tccaccagtg cctttggaca gccgtgcctg ttcgccgctc
2461 tccctaagtc tgattctcat cgaggcccct cgcttctatg actgtgcttg cagaagagta
2521 aacactctcg gatgccgctg tcctggggga gcccgcggga gcctgtgaat gttgatacga
2581 gctggccagt cctgggccca gctcacttgt ccagctacct gccaggtggc tttcactgtg
2641 tttaaaatac attgcattcc aagctggtcc cctctgtgta tcactctact gagaaatcct
2701 gcctagtgtg ttttgggatg tgtcctagca tttacaagaa aatgaaaagc gtcctcttaa
2761 ttggcacccg aatgttgctg tggctcagtc acatatccca gggccctcgt cccgaggccg
2821 tgctgccccg agccccgagc ccctctgcag ctcacccttg gcttgttttc cgcaaacccg
2881 gtaaacgcaa gcccttgggg cagatgcaga agcagaagag ggaggggaaa cctgcctctg
2941 ggtcaccctg ttagcacagc gttctcatcg ggagacagca tggaactctc tctcgcagtg
3001 ctcgaggctg tgtgtcagtg tttgctgggc ttgtggctcc ttttttggct ggataaagaa
3061 gtcgctgttt ttgtactgct tctgtggctc ttcacagacc tcacggatgt gaccggagat
3121 gagtgccgat gaccacgttt taaaggagaa agagagctcc tggtggggcc ctcggggtgg
3181 tctcaggtcc catttgcagt ctgcaacagt gacgcgcagc ccggtccgga gcgtggtgag
3241 ctttgtttgc cttctgggtc agctttcgct gtgtctcctg tgtgtgttag aatccagagc
3301 ccagaggaag tgcaagcggg tcctccgcca acggggagag cctcttcgcg gcgctgttgg
3361 cgacagcagc gctgtgattc gcgtagcagg ggagttgttt gaaacacctt cctgagtagt
3421 ccggccttgt caatgagtgc ttgttttcct ttaaacagtc tgacatattt actcgtcact
3481 ttcaaaccag aagcatgaga ggaaggagat attgtggggt ccgtttaact cgatagaaag
3541 cgcaggggga tggcccccgg cgcgggctct tgacccgctc agcgctgacc ccaccgccct
3601 ggccgaggca cttggccttg ctgagctgga cttcctcctc ctcctcctca tgaccggggt
3661 gaattagaac gtttttaaag acaccccctt ccaaattctg taacacattg taattggaga
3721 agaaggaaac tctgcaaggc taaactgtca ttcacaactt ggctacacat agactctagt
3781 cagttttgtc tccagaacct taggcttttg tattttttaa ttttaatttc actgttaatc
3841 cttattgtct tttttattaa gatgttggaa aagcaggagg tagttgtgcc tcaattattg
3901 caaaaatgta acaataaagt tcctcaaaat aagatctgtt cctcatagct atactgtgta
3961 cacataagac.gcatataggg ttttactgaa atctattttt aactcttatg ttcgtagaga
4021 aattgtttca aggattttga gtcataggtc tgtaatttat agagatctct agaattctta
4081 ttgtaatttt cctacttctt tgataaaaga aaaataagtc agattgttaa ctccaagatt
4141 gaaaaaaaaa actcttgaaa gaagattatt agttgtaact aatttagggg ttctgggcac
4201 agacatctaa cctggtattg taaggcagag gctcccattg gaatggtagt ggtccgggtc
4261 agttgttcat ggtgtaagct ttgcacagtg tattaacatt gggagggtct ggcttgaaaa
4321 tttggccacc ctcagcctct gaatgtttat taaaataaat ttagtctttc tttgcttaat
4381 ataaaaaaaa aaaaaaaa
```

See also NM_{—}007353 (posted on Feb. 10, 2008). The bold-faced region refers to a RNAi targeting region.)

```
Amino acid seuuence of human Gα₁₂
    1 msgvvrtlsr cllpaeagga rerragsgar daerearrrs rdidallare rravrrlvki
  61 lllgagesgk stflkqmrii hgrefdqkal lefrdtifdn ilkgsrvlvd ardklgipwq
121 ysenekhgmf lmafenkagl pvepatfqly vpalsalwrd sgireafsrr sefqlgesvk
181 yfldnldrig qlnyfpskqd illarkatkg ivehdfvikk ipfkmvdvgg qrsqrqkwfq
241 cfdgitsilf mvssseydqv lmedrrtnrl vesmnifeti vnnklffnvs iilflnkmdl
301 lvekvktvsi kkhfpdfrgd phrledvqry lvqcfdrkrr nrskplfhhf ttaidtenvr
361 fvfhavkdti lqenlkdiml q
Nucleotide sequence of human IOGAPI
      1 GACGGCACGGGGCGGGGCCTCGGGGACCCCGGCAAGCCCGCGCACTTGGCAGGAGCTGTA
    61 GCTACCGCCGTCCGCGCCTCCAAGGTTTCACGGCTTCCTCAGCAGAGACTCGGGCTCGTC
  121 CGCCATGTCCGCCGCAGACGAGGTTGACGGGCTGGGCGTGGCCCGGCCGCACTATGGCTC
  181 TGTCCTGGATAATGAGACTTACTGCAGAGGAGATGGATGAAAGGAGACGTCACAGAACGT
  241 GGCTTATGAGTACCTTTGTCATTTGGAAGAAGCGAAGAGGTGGATGGAAGCATGCCTAGG
  301 GGAAGATCTGCCTCCCACCACAGAACTGGAGGAGGGGCTTAGGAATGGGGTCTACCTTGC
  361 CAAACTGGGGAACTTCTTCTCTCCCAAAGTAGTGTCCCTGAAAAAAATCTATGATCGAGA
  421 ACAGACCAGATACAAGGCGACTGGCCTCCACTTTAGACACACTGATAATGTGATTCAGTG
  481 GTTGAATGCCATGGATGAGATTGGATTGCCTAAGATTTTTTACCCAGAAACTACAGATAT
  541 CTATGATCGAAAGAACATGCCAAGATGTATCTACTGTATCCATGCACTCAGTTTGTACCT
  601 GTTCAAGCTAGGCCTGGCCCCTCAGATTCAAGACCTATATGGAAAGGTTGACTTCACAGA
  661 AGAAGAAATCAACAACATGAAGACTGAGTTGGAGAAGTATGGCATCCAGATGCCTGCCTT
  721 TAGCAAGATTGGGCGCATCTTGGCTAATGAACTGTCAGTGGATGAAGCCGCATTACATGC
  781 TGCTGTTATTGCTATTAATGAAGCTATTGACCGTAGAATTCCAGCCGACACATTTGCAGC
  841 TTTGAAAAATCCGAATGCCATGCTTGTAAATCTTGAAGAGCCCTTGGCATCCACTTACCA
  901 GGATATACTTTACCAGGCTAAGCAGGACAAAATGACAAATGCTAAAAACAGGACAGAAAA
  961 CTCAGAGAGAGAAAGAGATGTTTATGAGGAGCTGCTCACGCAAGCTGAAATTCAAGGCAA
1021 TATAAACAAAGTCAATACATTTTCTGCATTAGCAAATATCGACCTGGCTTTAGAACAAGG
1081 AGATGCACTGGCCTTGTTCAGGGCTCTGCAGTCACCAGCCCTGGGGCTTCGAGGACTGCA
1141 GCAACAGAATAGCGACTGGTACTTGAAGCAGCTCCTGAGTGATAAACAGCAGAAGAGACA
1201 GAGTGGTCAGACTGACCCCCTGCAGAAGGAGGAGCTGCAGTCTGGAGTGGATGCTGCAAA
1261 CAGTGCTGCCCAGCAATATCAGAGAAGATTGGCAGCAGTAGCACTGATTAATGCTGCAAT
1321 CCAGAAGGGTGTTGCTGAGAAGACTCTTTTGGAACTGATGAATCCCGAAGCCCAGCTGCC
1381 CCAGGTGTATCCATTTGCCGCCGATCTCTATCAGAAGGAGCTGGCTACCCTGCAGCGACA
1441 AAGTCCTGAACATAATCTCACCCACCCAGAGCTCTCTGTCGCAGTGGAGATGTTGTCATC
1501 GGTGGCCCTGATCAACAGGGCATTGGAATCAGGAGATGTGAATACAGTGTGGAAGCAATT
1561 GAGCAGTTCAGTTACTGGTCTTACCAATATTGAGGAAGAAAACTGTCAGAGGTATCTCGA
1621 TGAGTTGATGAAACTGAAGGCTCAGGCACATGCAGAGAATAATGAATTCATTACATGGAA
1681 TGATATCCAAGCTTGCGTGGACCATGTGAACCTGGTGGTGCAAGAGGAACATGAGAGGAT
1741 TTTAGCCATTGGTTTAATTAATGAAGCCCTGGATGAAGGTGATGCCCAAAAGACTCTGCA
1801 GGCCCTACAGATTCCTGCAGCTAAACTTGAGGGAGTCCTTGCAGAAGTGGCCCAGCATTA
1861 CCAAGACACGCTGATTAGAGCGAAGAGAGAGAAAGCCCAGGAAATCCAGGATGAGTCAGC
1921 TGTGTTATGGTTGGATGAAATTCAAGGTGGAATCTGGCAGTCCAACAAAGACACCCAAGA
1981 AGCACAGAAGTTTGCCTTAGGAATCTTTGCCATTAATGAGGCAGTAGAAAGTGGTGATGT
2041 TGGCAAAACACTGAGTGCCCTTCGCTCCCCTGATGTTGGCTTGTATGGAGTCATCCCTGA
2101 GTGTGGTGAAACTTACCACAGTGATCTTGCTGAAGCCAAGAAGAAAAAACTGGCAGTAGG
2161 AGATAATAACAGCAAGTGGGTGAAGCACTGGGTAAAAGGTGGATATTATTATTACCACAA
2221 TCTGGAGACCCAGGAAGGAGGATGGGATGAACCTCCAAATTTTGTGCAAAATTCTATGCA
2281 GCTTTCTCGGGAGGAGATCCAGAGTTCTATGTCTGGGGTGACTGCCGCATATAACCGAGA
2341 ACAGCTGTGGCTGGCCAATGAAGGCCTGATCACCAGGCTGCAGGCTCGCTGCCGTGGATA
2401 CTTAGTTCGACAGGAATTCCGATCCAGGATGAATTTCCTGAAGAAACAAATCCCTGCCAT
2461 CACCTGCATTCAGTCACAGTGGAGAGGATACAAGCAGAAGAAGGCATATCAAGATCGGTT
2521 AGCTTACCTGCGCTCCCACAAAGATGAAGTTGTAAAGATTCAGTCCCTGGCAAGGATGCA
2581 CCAAGCTCGAAAGCGCTATCGAGATCGCCTGCAGTACTTCCGGGACCATATAAATGACAT
2641 TATCAAAATCCAGGCTTTTATTCGGGCAAACAAAGCTCGGGATGACTACAAGACTCTCAT
2701 CAATGCTGAGGATCCTCCTATGGTTGTGGTCCGAAAATTTGTCCACCTGCTGGACCAAAG
2761 TGACCAGGATTTTCAGGAGGAGCTTGACCTTATGAAGATGCGGGAAGAGGTTATCACCCT
2821 CATTCGTTCTAACCAGCAGCTGGAGAATGACCTCAATCTCATGGATATCAAAATTGGACT
2881 GCTAGTGAAAAATAAGATTACGTTGCAGGATGTGGTTTCCCACAGTAAAAAACTTACCAA
2941 AAAAAATAAGGAACAGTTGTCTGATATGATGATGATAAATAAACAGAAGGGAGGTCTCAA
3001 GGCTTTGAGCAAGGAGAAGAGAGAGAAGTTGGAAGCTTACCAGCACCTGTTTTATTTATT
3061 GCAAACCAATCCCACCTATCTGGCCAAGCTCATTTTTCAGATGCCCCAGAACAAGTCCAC
3121 CAAGTTCATGGACTCTGTAATCTTCACACTCTACAACTACGCGTCCAACCAGCGAGAGGA
3181 GTACCTGCTCCTGCGGCTCTTTAAGACAGCACTCCAAGAGGAAATCAAGTCGAAGGTAGA
3241 TCAGATTCAAGAGATTGTGACAGGAAATCCTACGGTTATTAAAATGGTTGTAAGTTTCAA
3301 CCGTGGTGCCCGTGGCCAGAATGCCCTGAGACAGATCTTGGCCCCAGTCGTGAAGGAAAT
3361 TATGGATGACAAATCTCTCAACATCAAAACTGACCCTGTGGATATTTACAAATCTTGGGT
3421 TAATCAGATGGAGTCTCAGACAGGAGAGGCAAGCAAACTGCCCTATGATGTGACCCCTGA
3481 GCAGGCGCTAGCTCATGAAGAAGTGAAGACACGGCTAGACAGCTCCATCAGGAACATGCG
3541 GGCTGTGACAGACAAGTTTCTCTCAGCCATTGTCAGCTCTGTGGACAAAATCCCTTATGG
3601 GATGCGCTTCATTGCCAAAGTGCTGAAGGACTCGTTGCATGAGAAGTTCCCTGATGCTGG
3661 TGAGGATGAGCTGCTGAAGATTATTGGTAACTTGCTTTATTATCGATACATGAATCCAGC
3721 CATTGTTGCTCCTGATGCCTTTGACATCATTGACCTGTCAGCAGGAGGCCAGCTTACCAC
3781 AGACCAACGCCGAAATCTGGGCTCCATTGCAAAAATGCTTCAGCATGCTGCTTCCAATAA
3841 GATGTTTCTGGGAGATAATGCCCACTTAAGCATCATTAATGAATATCTTTCCCAGTCCTA
3901 CCAGAAATTCAGACGGTTTTTCCAAACTGCTTGTGATGTCCCAGAGCTTCAGGATAAATT
3961 TAATGTGGATGAGTACTCTGATTTAGTAACCCTCACCAAACCAGTAATCTACATTTCCAT
4021 TGGTGAAATCATCAACACCCACACTCTCCTGTTGGATCACCAGGATGCCATTGCTCCGGA
4081 GCACAATGATCCAATCCACGAACTGCTGGACGACCTCGGCGAGGTGCCCACCATCGAGTC
4141 CCTGATAGGGGAAAGCTCTGGCAATTTAAATGACCCAAATAAGGAGGCACTGGCTAAGAC
4201 GGAAGTGTCTCTCACCCTGACCAACAAGTTCGACGTGCCTGGAGATGAGAATGCAGAAAT
4261 GGATGCTCGAACCATCTTACTGAATACAAAACGTTTAATTGTGGATGTCATCCGGTTCCA
4321 GCCAGGAGAGACCTTGACTGAAATCCTAGAAACACCAGCCACCAGTGAACAGGAAGCAGA
4381 ACATCAGAGAGCCATGCAGAGACGTGCTATCCGTGATGCCAAAACACCTGACAAGATGAA
4441 AAAGTCAAAATCTGTAAAGGAAGACAGCAACCTCACTCTTCAAGAGAAGAAAGAGAAGAT
4501 CCAGACAGGTTTAAAGAAGCTAACAGAGCTTGGAACCGTGGACCCAAAGAACAAATACCA
4561 GGAACTGATCAACGACATTGCCAGGGATATTCGGAATCAGCGGAGGTACCGACAGAGGAG
4621 AAAGGCCGAACTAGTGAAACTGCAACAGACATACGCTGCTCTGAACTCTAAGGCCACCTT
4681 TTATGGGGAGCAGGTGGATTACTATAAAAGCTATATCAAAACCTGCTTGGATAACTTAGC
4741 CAGCAAGGGCAAAGTCTCCAAAAAGCCTAGGGAAATGAAAGGAAAGAAAAGCAAAAAGAT
4801 TTCTCTGAAATATACAGCAGCAAGACTACATGAAAAAGGAGTTCTTCTGGAAATTGAGGA
4861 CCTGCAAGTGAATCAGTTTAAAAATGTTATATTTGAAATCAGTCCAACAGAAGAAGTTGG
4921 AGACTTCGAAGTGAAAGCCAAATTCATGGGAGTTCAAATGGAGACTTTTATGTTACATTA
4981 TCAGGACCTGCTGCAGCTACAGTATGAAGGAGTTGCAGTCATGAAATTATTTGATAGAGC
5041 TAAAGTAAATGTCAACCTCCTGATCTTCCTTCTCAACAAAAAGTTCTACGGGAAGTAATT
5101 GATCGTTTGCTGCCAGCCCAGAAGGATGAACCAAAGAAGCACCTCACAGCTCCTTTCTAG
5161 GTCCTTCTTTCCTCATTGGAAGCAAAGACCTAGCCAACAACAGCACCTCAATCTGATACA
5221 CTCCCCATGCCACATTTTTAACTCCTCTCGCTCTGATGGGACATTTGTTACCCTTTTTTC
5281 ATAGTGAAATTGTGTTTCAGGCTTAGTCTGACCTTTCTGGTTTCTTCATTTTCTTCCATT
5341 ACTTAGGAAAGAGTGGAAACTCCACTAAAATTTCTCTGTGTTGTTACAGTCTTAGAGGTT
5401 GCAGTACTATATTGTAAGCTTTGGTGTTTGTTTAATTAGCAATAGGGATGGTAGGATTCA
5461 ATGTGTGTCATTTAGAAGTGGAAGCTATTAGCACCAATGACATAAATACATACAAGACA
5521 CACAACTAAAATGTCATGTTATTAACAGTTATTAGGTTGTCATTTAAAAATAAAGTTCCT
5581 TTATATTTCTGTCCCATCAGGAAAACTGAAGGATATGGGGAATCATTGGTTATCTTCCAT
5641 TGTGTTTTTCTTTATGGACAGGAGCTAATGGAAGTGACAGTCATGTTCAAAGGAAGCATT
5701 TCTAGAAAAAAGGAGATAATGTTTTTAAATTTCATTATCAAACTTGGGCAATTCTGTTTG
5761 TGTAACTCCCCGACTAGTGGATGGGAGAGTCCCATTGCTAAAATTCAGCTACTCAGATAA
5821 ATTCAGAATGGGTCAAGGCACCTGCCTGTTTTTGTTGGTGCACAGAGATTGACTTGATTC
5881 AGAGAGACAATTCACTCCATCCCTATGGCAGAGGAATGGGTTAGCCCTAATGTAGAATGT
5941 CATTGTTTTTAAAACTGTTTTATATCTTAAGAGTGCCTTATTAAAGTATAGATGTATGTC
6001 TTAAAATGTGGGTGATAGGAATTTTAAAGATTTATATAATGCATCAAAAGCCTTAGAATA
6061 AGAAAAGCTTTTTTTAAATTGCTTTATCTGTATATCTGAACTCTTGAAACTTATAGCTAA
6121 AACACTAGGATTTATCTGCAGTGTTCAGGGAGATAATTCTGCCTTTAATTGTCTAAAACA
6181 AAAACAAAACCAGCCAACCTATGTTACACGTGAGATTAAAACCAATTTTTTCCCCATTTT
6241 TTCTCCTTTTTTCTCTTGCTGCCCACATTGTGCCTTTATTTTATGAGCCCCAGTTTTCTG
6301 GGCTTAGTTTAAAAAAAAAATCAAGTCTAAACATTGCATTTAGAAAGCTTTTGTTCTTGG
6361 ATAAAAAGTCATACACTTTAAAAAAAAAAAAAACTTTTTCCAGGAAAATATATTGAAATC
6421 ATGCTGCTGAGCCTCTATTTTCTTTCTTTGATGTTTTGATTCAGTATTCTTTTATCATAA
6481 ATTTTTAGCATTTAAAAATTCACTGATGTACATTAAGCCAATAAACTGCTTTAATGAATA
6541 ACAAACTATGTAGTGTGTCCCTATTATAAATGCATTGGAGAAGTATTTTTATGAGACTCT
6601 TTACTCAGGTGCATGGTTACAGCCCACAGGGAGGCATGGAGTGCCATGGAAGGATTCGCC
6661 ACTACCCAGACCTTGTTTTTTGTTGTATTTTGCAAGACAGGTTTTTTAAAGAAACATTTT
6721 CCTCAGATTAAAAGATGATGCTATTACAACTAGCATTGCCTCAAAAACTGGGACCAACCA
6781 AAGTGTGTCAACCCTGTTTCCTTAAAAGAGGCTATGAATCCCAAAGGCCACATCCAAGAC
6841 AGGCAATAATGAGCAGAGTTTACAGCTCCTTTAATAAAATGTGTCAGTAATTTTAAGGTT
6901 TATAGTTCCCTCAACACAATTGCTAATGCAGAATAGTGTAAAATGCGCTTCAAGAATGTT
6961 GATGATGATGATATAGAATTGTGGCTTTAGTAGCACAGAGGATGCCCCAACAAACTCATG
7021 GCGTTGAAACCACACAGTTCTCATTACTGTTATTTATTAGCTGTAGCATTCTCTGTCTCC
7081 TCTCTCTCCTCCTTTGACCTTCTCCTCGACCAGCCATCATGACATTTACCATGAATTTAC
7141 TTCCTCCCAAGAGTTTCGACTGCCCGTCAGATTGTTGCTGCACATAGTTGCCTTTGTATC
7201 TCTGTATGAAATAAAAGGTCATTTGTTCATGTT
Amino acid sequence of human IOGAP1
      1 MSAADEVDGLGVARPHYGSVLDNERLTAEEMDERRRQNVAYEYLCHLEEAKRWMEACLGE
    61 DLPPTTELEEGLRNGVYLAKLGNFFSPKVVSLKKIYDREQTRYKATGLHFRHTDNVIQWL
  121 NAMDEIGLPKIFYPETTDIYDRKNMPRCIYCIHALSLYLFKLGLAPQIQDLYGKVDFTEE
  181 EINNMKTELEKYGIQMPAFSKIGGILANELSVDEAALHAAVIAINEAIDRRIPADTFAAL
  241 KNPNAMLVNLEEPLASTYQDILYQAKQDKMTNAKNRTENSERERDVYEELLTQAEIQGNI
  301 NKVNTFSALANIDLALEQGDALALFRALQSPALGLRGLQQQNSDWYLKQLLSDKQQKRQS
  361 GQTDFLQKEELQSGVDAANSAAQQYQRRLAAVALINAAIQKGVAEKTVLELNNPEAQLPQ
  421 VYPFAADLYQKELATLQRQSPEHNLTHPELSVAVEMLSSVALINPALESGDVNTVWKQLS
  481 SSVTGLTNIEEENCQRYLDELMKLKAQAHAENNEFITWNDIQACVDHVNLVVQEEHERIL
  541 AIGLINEALDEGDAQKTLQALQIPAAKLEGVLAEVAQHYQDTLIRAKREKAQEIQDESAV
  601 LWLDEIQGGIWQSNKDTQEAQKFALGIFAINEAVESGDVGKTLSALRSPDVGLYGVIPEC
  661 GETYHSDLAEAKKKKLAVGDNNSKWVKHWVKGGYYYYHNLETQEGGWDEPPNFVQNSMQL
  721 SREEIQSSISGVTAAYNREQLWLANEGLITRLQARCRGYLVRQEFRSRMNFLKKQIPAIT
  781 CIQSQWRGYKQKKAYQDRLAYLRSHKDEVVKIQSLARMHQARKRYRDRLQYFRDHINDII
  841 KIQAFIRANKARDDYKTLINAEDPPMVVVRKFVHLLDQSDQDFQEELDLMKMREEVITLI
  901 RSNQQLENDLNLMDIKIGLLVKNKITLQDVVSHSKKLTKKNKEQLSDMMMINKQKGGLKA
  961 LSKEKREKLEAYQHLFYLLQTNPTYLAKLIFQMPQNKSTKFMDSVIFTLYNYASNQREEY
1021 LLLRLFKTALQEEIKSKVDQIQEIVTGNPTVIKMVVSFNRGARGQNALRQILAPVVKEIM
1081 DDKSLNIKTDPVDIYKSWVNQMESQTGEASKLPYDVTPEQALAHEEVKTRLDSSIRNMRA
1141 VTDKFLSAIVSSVDKIPYGMRFIAKVLKDSLHEKFPDAGEDELLKIIGNLLYYRYMNPAI
1201 VAPDAFDIIDLSAGGQLTTDQRRNLGSIAKMLQHAASNKMFLGDNAHLSIINEYLSQSYQ
1261 KFRRFFQTACDVPELQDKFNVDEYSDLVTLTKPVIYISIGEIINTHTLLLDHQDAIAPEH
1321 NDPIHELLDDLGEVPTIESLIGESSGNLNDPNKEALAKTEVSLTLTNKFDVPGDENAEM
1381 ARTILLNTKRLIVDVIRFQPGETLTEILETPATSEQEAEHQRAMQRRAIRDAKTPDKMKK
1441 SKSVKEDSNLTLQEKKEKIQTGLKKLTELGTVDPKNKYQELINDIARDIRNQRRYRQRRK
1501 AELVKLQQTYAALNSKATFYGEQVDYYKSYIKTCLDNLASKGKVSKKPREMKGKKSKKIS
1561 LKYTAARLHEKGVLLEIEDLQVNQFKNVIFEISPTEEVGDFEVKAKFMGVQMETFMLHYQ
1621 DLLQLQYEGVAVMKLFDRAKVNVNLLIFLLNKKFYGK
```

In a preferred example, the compound is a double-strand RNA (dsRNA) that inhibits the expression of any of the genes mentioned above via RNA interference. RNA interference (RNAi) is a process in which a dsRNA directs homologous sequence-specific degradation of messenger RNA. In mammalian cells, RNAi can be triggered by 21-nucleotide duplexes of small interfering RNA (siRNA) without activating the host interferon response. As this process represses the expression of one of the three innate immunity receptors described herein, it can be used to treat influenza virus infection.

In one example, the dsRNA can be a siRNA that inhibits the expression of the Gα₁₂ gene, e.g., targeting the nucleotide sequence 5′-GCGACACCATCTTCGACAACA-3′ in a Gα₁₂ gene (see the bold-faced region in the human Gα₁₂ gene sequence shown above). See Shin et al., Proc. Natl. Acad. Sci. U.S.A. (2006) 103(37):13759-13764. Examples of siRNAs that suppress Gα₁₂ gene expression include, but are not limited to, Gα₁₂ siRNAs provided by Dharmacon (product number L-008435-00), which include four siRNAs each having the nucleotide sequence 5′-GGGAGUCGGUGAAGUACUUUU-3′,5′-GGAUCGGCCAGCUGAAUUAUU-3′, 5′-GGAAAGCCACCAAGGGAAUUU-3′, or 5′-GAGAUAAGCUUGGCAUUCCUU-3′.

In another example, the dsRNA is a siRNA that inhibits the expression of the IQGAP1 gene. Examples include, but are not limited to, 5′-GAACGUGGCUUAUGAGUACUU-3′, 5′-GCAGGUGGAUUACUAUAAAUU-3′,5′-CGAACCAUCUUACUGAAUAUU-3′, and 5′-CAAUUGAGCAGUUCAGUUAUU-3′.

A dsRNA can be synthesized by methods known in the art. See, e.g., Caruthers et al., 1992, Methods in Enzymology 211, 3-19, Wincott et al., 1995, Nucleic Acids Res. 23, 2677-2684, Wincott et al., 1997, Methods Mol. Bio. 74, 59, Brennan et al., 1998, Biotechnol Bioeng., 61, 33-45, and Brennan, U.S. Pat. No. 6,001,311. It can also be transcribed from an expression vector and isolated using standard techniques.

The dsRNA or vector as described above can be delivered to a virus target cell by methods, such as that described in Akhtar et al., 1992, Trends Cell Bio. 2, 139. For example, it can be introduced into cells using liposomes, hydrogels, cyclodextrins, biodegradable nanocapsules, or bioadhesive microspheres. Alternatively, the dsRNA or vector can be locally delivered by direct injection or by use of an infusion pump. Other approaches include employing various transport and carrier systems, for example through the use of conjugates and biodegradable polymersone or more small interfering RNAs.

The agent used in the method for inhibiting NPC invasion as described herein also can be an antibody that specifically binds to a protein involved in the Gα₁₂ signaling pathway (see FIG. 1) and blocks protein function, or specifically binds to IQGAP1 and blocks its function.

Methods of making monoclonal and polyclonal antibodies and fragments thereof in animals are known in the art. See, for example, Harlow and Lane, (1988) Antibodies: A Laboratory Manual, Cold Spring Harbor Laboratory, New York. The term “antibody” includes intact molecules, e.g., monoclonal antibody, polyclonal antibody, chimeric antibody, humanized antibody, as well as fragments thereof, e.g., Fab, F(ab′)₂, Fv, scFv (single chain antibody), and dAb (domain antibody; Ward, et. al. (1989) Nature, 341, 544).

In general, to produce antibodies against a peptide, the peptide can be coupled to a carrier protein, such as KLH, mixed with an adjuvant, and injected into a host animal. Antibodies produced in the animal can then be purified by peptide affinity chromatography. Commonly employed host animals include rabbits, mice, guinea pigs, and rats. Various adjuvants that can be used to increase the immunological response depend on the host species and include Freund's adjuvant (complete and incomplete), mineral gels such as aluminum hydroxide, CpG, surface-active substances such as lysolecithin, pluronic polyols, polyanions, peptides, oil emulsions, keyhole limpet hemocyanin, and dinitrophenol. Useful human adjuvants include BCG (bacille Calmette-Guerin) and Corynebacterium parvum.

Polyclonal antibodies, heterogeneous populations of antibody molecules, are present in the sera of the immunized subjects. Monoclonal antibodies, homogeneous populations of antibodies to a polypeptide of this invention, can be prepared using standard hybridoma technology (see, for example, Kohler et al. (1975) Nature 256, 495; Kohler et al. (1976) Eur. J. Immunol. 6, 511; Kohler et al. (1976) Eur J Immunol 6, 292; and Hammerling et al. (1981) Monoclonal Antibodies and T Cell Hybridomas, Elsevier, N.Y.). In particular, monoclonal antibodies can be obtained by any technique that provides for the production of antibody molecules by continuous cell lines in culture such as described in Kohler et al. (1975) Nature 256, 495 and U.S. Pat. No. 4,376,110; the human B-cell hybridoma technique (Kosbor et al. (1983) Immunol Today 4, 72; Cole et al. (1983) Proc. Natl. Acad. Sci. USA 80, 2026, and the EBV-hybridoma technique (Cole et al. (1983) Monoclonal Antibodies and Cancer Therapy, Alan R. Liss, Inc., pp. 77-96). Such antibodies can be of any immunoglobulin class including IgG, IgM, IgE, IgA, IgD, and any subclass thereof. The hybridoma producing the monoclonal antibodies of the invention may be cultivated in vitro or in vivo. The ability to produce high titers of monoclonal antibodies in vivo makes it a particularly useful method of production. In addition, techniques developed for the production of “chimeric antibodies” can be used. See, e.g., Morrison et al. (1984) Proc. Natl. Acad. Sci. USA 81, 6851; Neuberger et al. (1984) Nature 312, 604; and Takeda et al. (1984) Nature 314:452. A chimeric antibody is a molecule in which different portions are derived from different animal species, such as those having a variable region derived from a murine monoclonal antibody and a human immunoglobulin constant region. Alternatively, techniques described for the production of single chain antibodies (U.S. Pat. Nos. 4,946,778 and 4,704,692) can be adapted to produce a phage library of single chain Fv antibodies. Single chain antibodies are formed by linking the heavy and light chain fragments of the Fv region via an amino acid bridge. Moreover, antibody fragments can be generated by known techniques. For example, such fragments include, but are not limited to, F(ab′)₂ fragments that can be produced by pepsin digestion of an antibody molecule, and Fab fragments that can be generated by reducing the disulfide bridges of F(ab′)₂ fragments. Antibodies can also be humanized by methods known in the art. For example, monoclonal antibodies with a desired binding specificity can be commercially humanized (Scotgene, Scotland; and Oxford Molecular, Palo Alto, Calif.). Fully human antibodies, such as those expressed in transgenic animals are also features of the invention (see, e.g., Green et al. (1994) Nature Genetics 7, 13; and U.S. Pat. Nos. 5,545,806 and 5,569,825).

Antibodies thus prepared can be tested via well-established in vivo or in vitro systems for their activity of inhibiting the Gα₁₂ signaling pathway. Those showing positive results can be used for inhibiting NPC invasion.

In another example, the agent used in the method for inhibiting NPC invasion as described herein is a small molecule (organic or inorganic) that suppresses the Gα₁₂ signaling pathway, i.e., inhibiting the activity of a protein involved in this pathway or down-regulating the expression level of a gene involved in the pathway. Such small molecules include, but are not limited to, Y-27632 and dimethyl BAPTA. See Dorsam et al., J. Bio. Chem. (2002) 277(49):47588-47595. Such a small molecule can also be screened by any method known in the art, e.g., platelet aggregation assay. See, e.g., Dorsam et al.

In one in vivo approach, a therapeutic composition, containing any of the above-described agents and a pharmaceutically acceptable carrier, is administered to a subject via a conventional route. The carrier in the therapeutic composition must be “acceptable” in the sense that it is compatible with the active ingredient of the composition, and preferably, capable of stabilizing the active ingredient and not deleterious to the subject to be treated. The agent can be dissolved or suspended in the carrier (e.g., physiological saline) and administered orally or by intravenous infusion, or injected or implanted subcutaneously, intramuscularly, intrathecally, intraperitoneally, intrarectally, intravaginally, intranasally, intragastrically, intratracheally, or intrapulmonarily.

The dosage required depends on the choice of the route of administration; the nature of the formulation; the nature of the subject's illness; the subject's size, weight, surface area, age, and sex; other drugs being administered; and the judgment of the attending physician. Suitable dosages are in the range of 0.01-100.0 mg/kg. Wide variations in the needed dosage are to be expected in view of the variety of compositions available and the different efficiencies of various routes of administration. For example, oral administration would be expected to require higher dosages than administration by intravenous injection. Variations in these dosage levels can be adjusted using standard empirical routines for optimization as is well understood in the art. Encapsulation of the composition in a suitable delivery vehicle (e.g., polymeric microparticles or implantable devices) may increase the efficiency of delivery, particularly for oral delivery.

The just-described therapeutic composition can be formulated into dosage forms for different administration routes utilizing conventional methods. For example, it can be formulated in a capsule, a gel seal, or a tablet for oral administration. Capsules can contain any standard pharmaceutically acceptable materials such as gelatin or cellulose. Tablets can be formulated in accordance with conventional procedures by compressing mixtures of the composition with a solid carrier and a lubricant. Examples of solid carriers include starch and sugar bentonite. The composition can also be administered in a form of a hard shell tablet or a capsule containing a binder, e.g., lactose or mannitol, a conventional filler, and a tableting agent. The pharmaceutical composition can be administered via the parenteral route. Examples of parenteral dosage forms include aqueous solutions, isotonic saline or 5% glucose of the active agent, or other well-known pharmaceutically acceptable excipient. Cyclodextrins, or other solubilizing agents well known to those familiar with the art, can be utilized as pharmaceutical excipients for delivery of the therapeutic agent.

The efficacy of the agent for inhibiting NPC invasion, preferably contained in the therapeutic composition described above, can be evaluated both in vitro and in vivo. Based on the results, an appropriate dosage range and administration route can be determined.

Also within the scope of this invention is a method for screening a compound that inhibits NPC invasion by suppressing the Gα₁₂ signaling pathway. An example follows. NPC cells are incubated in the presence or absence of a test compound for a suitable time period. The activation level of the Gα₁₂ signaling pathway is then determined by, e.g., expression level (i.e., mRNA level or protein level) of a gene involved in the Gα₁₂ signaling pathway. If the activation of the Gα₁₂ signaling pathway in cells incubated with the test compound is down-regulated relative to that in cells free from the test compound, it indicates that the test compound suppresses the signal pathway. In other words, the test compound is a drug candidate for inhibiting NPC invasion.

Appendix II incorporated hereto shows genes that are differentially expressed in Gα₁₂-expressing cells and Gα₁₂ depleted cells. The expression level of these genes also can be used as a read out indicating the activation level of the Gα₁₂ signaling pathway in a cell.

Appendix III incorporated hereto shows the biological processes that are altered in nasopharyngeal carcinoma cells as compared with those in normal cells.

Alternatively, as cell morphology and mobility are associated with the activation level of the Gα₁₂ signaling pathway (see Example 3, below), they can be used as read-outs in the just-described screening method.

The above-mentioned test compound can be obtained from compound libraries, such as peptide libraries or peptoid libraries. The libraries can be spatially addressable parallel solid phase or solution phase libraries. See, e.g., Zuckermann et al. J. Med. Chem. 37, 2678-2685, 1994; and Lam Anticancer Drug Des. 12:145, 1997. Methods for the synthesis of compound libraries are well known in the art, e.g., DeWitt et al. PNAS USA 90:6909, 1993; Erb et al. PNAS USA 91:11422, 1994; Zuckermann et al. J. Med. Chem. 37:2678, 1994; Cho et al. Science 261:1303, 1993; Carrell et al. Angew Chem. Int. Ed. Engl. 33:2059, 1994; Carell et al. Angew Chem. Int. Ed. Engl. 33:2061, 1994; and Gallop et al. J. Med. Chem. 37:1233, 1994. Libraries of compounds may be presented in solution (e.g., Houghten Biotechniques 13:412-421, 1992), or on beads (Lam Nature 354:82-84, 1991), chips (Fodor Nature 364:555-556, 1993), bacteria (U.S. Pat. No. 5,223,409), spores (U.S. Pat. No. 5,223,409), plasmids (Cull et al. PNAS USA 89:1865-1869, 1992), or phages (Scott and Smith Science 249:386-390, 1990; Devlin Science 249:404-406, 1990; Cwirla et al. PNAS USA 87:6378-6382, 1990; Felici J. Mol. Biol. 222:301-310, 1991; and U.S. Pat. No. 5,223,409).

Without further elaboration, it is believed that one skilled in the art can, based on the above description, utilize the present invention to its fullest extent. The following specific embodiments are, therefore, to be construed as merely illustrative, and not limitative of the remainder of the disclosure in any way whatsoever. All publications cited herein are incorporated by reference.

### Example 1

### Genetic Markers for Diagnosing NPC

Nine primary cell lines derived from NPC patients and 32 primary nasopharyngeal epithelium (NPE) cells lines derived from healthy subjects were established by explant cell culture as described in, e.g., Peehl, Endocrine-Related Cancer 12:19-47, 2005; and Kino-oka et al., Adv Biochem Engin/Biotechnol 91:135-169, 2004. Briefly, fresh nasopharyngeal biopsies were cut to 1-2 mm explants and placed on top of an irradiated NIH/3T3 cell layer in DMEM Ham's-F12 medium (3:1) supplemented with 10% FBS, 1.8×10⁻⁴ M adenine, 0.4 μg/mL hydrocortisone, 5 μg/mL insulin, 10⁻¹⁰ M cholera toxin, 2×10⁻¹¹ M 3,3′,5-triiodo-L-thyronine, 5 μg/mL transferrin, 10 ng/mL epidermal growth factor, 10 μg/mL gentamicin and 2 μg/mL amphotericin B. After epithelial cells outgrew as visualized under a microscope, the explants were fed with defined keratinocyte serum-free medium (Invitrogen) to stimulate proliferation of epithelial cells. When the epithelial outgrowths reached about 5 mm², the explants were grown on collagen-coated culture vessels for subsequent passages before the onset of terminal differentiation. The third to ninth passages of preconfluent nasopharyngeal cells were harvested for RNA extraction and microarray experiments.

Gene expression profiles of the above-mentioned NPC and NPE primary cell lines, as well as 5 established NPC cell lines, were determined as follows. Total RNAs were extracted from each of the above-mentioned cell lines using the RNeasy kit (Qiagen). The RNAs obtained from the 32 NPE primary cell lines were pooled to produce a reference RNA sample. cDNAs, labeled with either Cyanine 3-dUTP or Cyanine 5-dUTP, were generated from the RNA samples obtained from the NPC primary/established cell lines and the reference RNA sample via methods known in the art.

The cDNAs thus obtained were then hybridized to a customized microarray chip containing 46,657 cDNAs that represent approximately 26,000 unigene clusters (IMAGE consortium). After washing the chip for a suitable number of times, the fluorescence signals remaining on the chip were detected with the GenePix 4000B scanner. The results were analyzed using the GenePix software package (Axon Instruments) or GenMAPP/MappFinder software (see Dahlquist et al., Nat. Genet. 2002, 31:19-20) to identify genes that were differentially expressed in NPC cells relative to NPE cells. Preferably, the raw results were normalized following the method described in Tseng et al., Nucleic Acids Res. 2001, 29:2549-2557. Appendices I and II, both in computer-readable form, show the genes that are differentially expressed in NPC patients versus in healthy controls and genes differentially expressed in Gα₁₂-depleted NPC cells, respectively. Appendix III, also in computer-readable form, shows the biological pathways that are altered in NPC patients.

A large number of genes were found to be either up-regulated or down-regulated in NPC cells (p≦0.05). See Appendices I and II. These genes are involved in various cellular structure/processes, including RNA processing, transcription, chromatin architecture, protein modification, macromolecule metabolism, organelle organization, and biogenesis.

Genes involved in G protein-coupled receptor (GPCR) signaling pathways were found to be differentially expressed in 11 out of 14 NPC cell lines. Among them, a number of over-expressed genes, e.g., Gα₁₂, ARHGEF12, RhoA, SLC9A1, ROCK1, PFN1, and JNK, were identified to be associated with the Gα₁₂ signaling pathway, using the Ingenuity pathway analysis and GenMAPP/MappFinder software packages.

The expression levels of Gα₁₂ in biopsy samples obtained from NPC patients who had neck lymph node metastasis, i.e., L.N.(+), and from those who had no lymph node metastasis, i.e., L.N.(−), were determined via quantitative real-time reverse-transcription PCR (QRT-PCR). Briefly, the QRT-PCR analysis was performed using a LightCycler PCR system (Roche) and the FastStart DNA Master.SYBR Green I Kit (Roche Applied Science). The expression levels of Gα₁₂ thus obtained were normalized against the expression levels of MAP4 in the same biopsy samples. The relative quantification was calculated using RelQuant software (Roche). The expression levels of Gα₁₂ in biopsy samples obtained from L.N.(+) were much higher than those in biopsy samples obtained from L.N.(−) (P<0.05). This result indicates that the expression level of Gα₁₂ correlates with the invasive/metastatic stage of NPC.

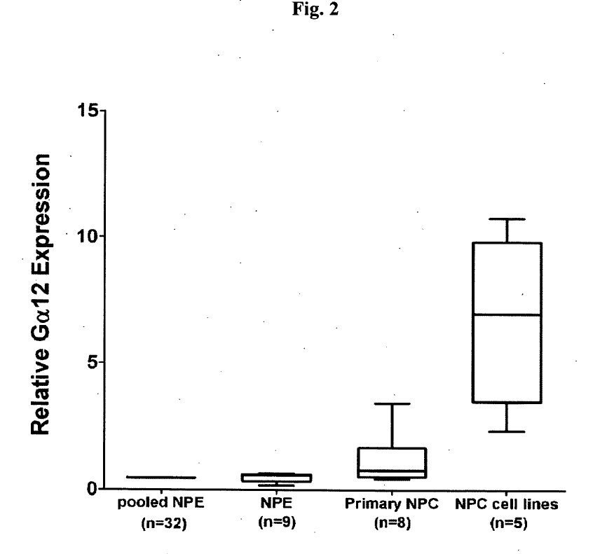

QRT-PCR was performed to examine the Gα₁₂ expression level in primary nasopharyngeal epithelium (NPE) cells, primary nasopharyngeal carcinoma (NPC) cells, and NPC cell lines. As shown in FIG. 2, the expression levels of Gα₁₂ in NPE cells are much lower than those in the NPC cells.

Overexpression of Gα₁₂ was also found to be associated with radioresistance of NPC cells. DNA constructs for expression wild-type Gα₁₂ and Gα₁₂ mutant Gα₁₂Q231L were introduced into NPC cells. The transfected cells were then subjected to γ-ray irradiation (6 Gy). The viability of these cells was examined afterwards. Results indicate that cells overepxressing either the wild-type Gα₁₂ or the mutant Gα₁₂Q231L are more resistant to irradiation than control cells.

### Example 2

### Determining Gα₁₂ Levels in Biopsy Samples by Immunostaining

The expression levels of Gα₁₂ were examined in 13 nasopharyngeal biopsy samples obtained from healthy controls, 6 nasopharyngeal biopsy samples from patients having different levels of dysplasia lesions, and 31 nasopharyngeal biopsy samples from NPC patients, following the method described in Chang et al., Cynecologic Oncology (1999), 73(1):62-71, using an anti-Gα₁₂ antibody (1:100; sc409, Santa Cruz Biotechnology, Inc.). Based on the percentages of positive cells, intensity scores “−”, “+”, “++”, and “+++” scoring, referring to negative or <20% positive cells, 21%-50% positive cells, 51%-70% positive cells, and >71% positive cells, respectively, were assigned to all biopsy samples examined in this study. The intensity scores of most NPE samples (12/13) are “−”, indicating that the expression of Gα₁₂ was barely detectable in these normal samples. Low to medium levels of Gα₁₂ expression were detected in samples containing dysplasic lesions at various severity (mild, moderate, and severe). On the other hand, strong Gα₁₂ immunoreactivity was detected in NPC samples. More specifically, of the 31 NPC biopsies, 58% (18/31) were scored “+++”, 35.5% (11/31) scored “++”, and 6.5% (2/31) scored “+” in tumor masses. Further, the expression level of Gα₁₂ was much higher in NPC tissues than in adjacent basal layer epithelium. These results indicate that the level of Gα₁₂ expression correlates with nasopharyngeal carcinoma (P<0.01). In other words, the level of Gα₁₂ is a marker for diagnosing NPC.

### Example 3

### Reduction of NPC Cancer Cell Mobility and Inhibition of NPC Cell Proliferation by Suppressing Gα₁₂ Expression

CNE1 cells and NPC-TW06 cells (two NPC-derived cell lines) were seeded at a density of 5×10⁴ cells/well in a 24-well plate 24 hours prior to transfection with Gα₁₂ siRNA (L-008435-00, Dharmacon; also described above) or a control siRNA (Dharmacon D-001810-01-05) using the DharmaFECT 1 reagent. The transfected cells were cultured for 1-3 days before subjected to the functional assays described below.

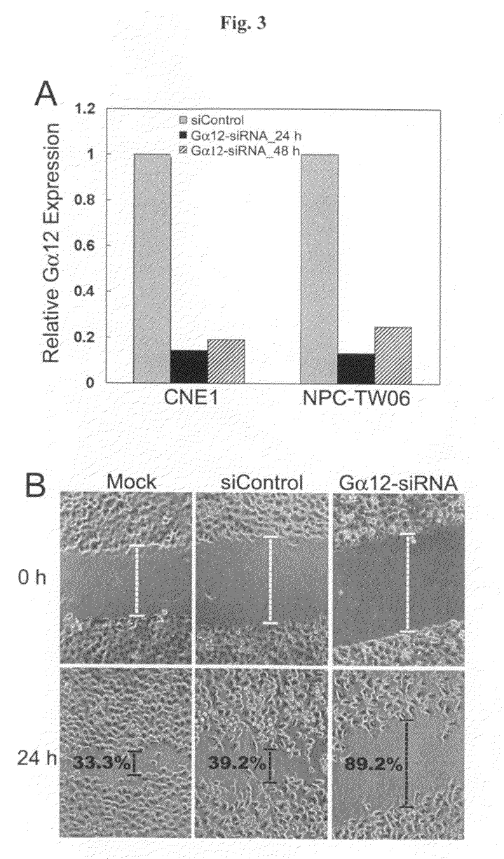

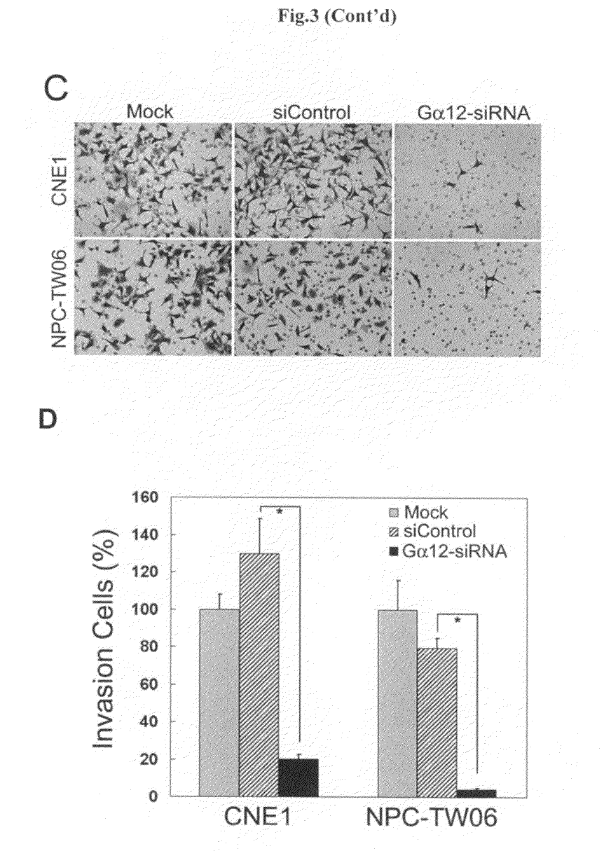

First, the Gα₁₂ mRNA and protein levels in both transfected CNE1 cells and NPC-TW06 cells were determined by QRT-PCR and western blot, respectively. As shown in FIG. 3, panel A, 24 hours after transfection, the expression levels Gα₁₂ in cells transfected with Gα₁₂ siRNA were about 80% lower than those in cells transfected with the control siRNA.

Second, a wound-healing assay was performed to test cell mobility. Briefly, forty-eight hours after transfection with Gα₁₂ siRNA or the control siRNA, NPC-TW06 cells were grown to confluence and carefully scratched with sterile 200 μl pipette tips to generate wounds. The widths of the wounds were photographed using a phase-contrast microscope at 0 and 24 hours post-scratching. All experiments were performed in triplicate. As shown in FIG. 3, panel B, the initial wound widths in non-transfected cells (Mock), cells transfected with the control siRNA, and cells transfected with the Gα₁₂ siRNA were very similar. 24 hours after scratching, the wound widths in mock cells and cells transfected with the control siRNA respectively were 33.3% and 39.2% of their initial wound widths. The wound width of cells transfected with Gα₁₂ siRNA, however, was 89.2% of its initial wound width. These data clearly indicate that inhibition of Gα₁₂ expression significantly reduces cell migration along the wound edges.

Similar results were observed in a matrigel invasion assay as described below. CNE1 and NPC-TW06 cells were transfected with the control siRNA and the Gα₁₂ siRNA as described above. 48 hours after transfection, the cells were harvested and resuspended in serum-free culture medium. The invasion capacities of the transfected cells were examined using an invasion chamber consisting of inserts containing 8 μm pore-size PET membrane coated with 80 μg Matrigel (BD, Biosciences), following manufacturer's instructions. After 30 hours-incubation at 37° C., the invaded cells were stained with 1% Gentian Violet. At least five distinct fields were counted for each duplicate. Relative numbers of invaded NPC cells were presented as mean±S.E. in triplicate. P value was determined by paired t test. Results thus obtained are shown in FIG. 3, panels C and D. Inhibition of Gα₁₂ expression significantly reduced NPC cell matrigel invasion (P<0.0001).

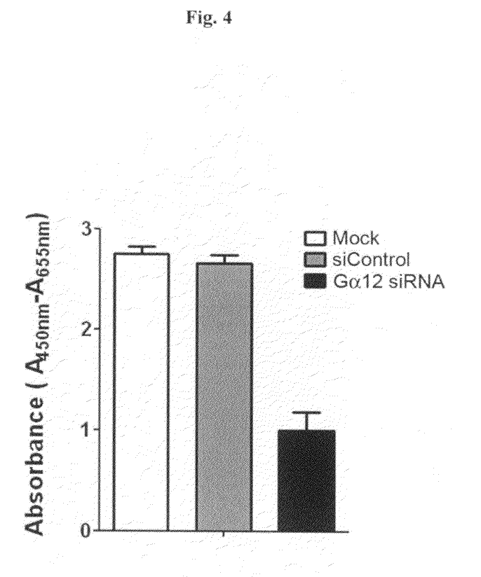

Next, the effect of inhibiting Gα₁₂ expression on NPC cell proliferation was tested. 3×10³/well NPC-TW06 cells seeded in a 96-well plate were transfected with either the control siRNA or the Gα₁₂ siRNA as described above. Cell proliferation was determined in triplicate by the WST-1 cell proliferation assay 72 hours post transfection. The plate was read using a Vmax microplate spectrophotometer. The results obtained from this assay show that Gα₁₂ siRNA inhibited NPC-TW06 cells proliferation 72 hours after transfection. See FIG. 4.

The anti-proliferation effect was confirmed by a flow cytometry assay for determining percentages of cells in different cell cycle phases. The cells transfected with the Gα₁₂ siRNA were accumulated at the G0/G1 while a substantial portion of the cells transfected with the control siRNA progressed to S phase. Overexpression of the wild-type Gα₁₂ and a Gα₁₂ mutant Gα₁₂Q231L resulted in increased percentages of cells in S and G₂/M phases, indicating that it promotes NPC cell proliferation.

Moreover, the effect of inhibiting Gα₁₂ expression on cell morphology was tested, using Rhodamine-Phalloidin to label F-actin. 48 hours after transfection, the cells transfected with the Gα₁₂ siRNA were flattened and multipolar, and had a larger cell-cell contact area.

Differently, both the mock cells and the cells transfected with the control siRNA were bipolar and had a small spindle-like appearance. In addition, F-actin bundles in the cells transfected with Gα₁₂ siRNA were more continuously aligned along cell edges than those in cells transfected with control siRNA, which were located at the bipolar ends of the cells. Since accumulation of actin at the migrating fronts contributes to cell mobility, these results also indicate that the dysregulation of Gα₁₂ alters actin dynamics, which in turn contribute to cell migration and invasion in NPC.

The levels of 95 differentially expressed genes that are involved in actin cytoskeleton signaling (see Table 1 below) were examined in both control NPC cells and in NPC cells transfected with Gα₁₂ siRNA by microarray. The results thus obtained were shown in Table 1 below:

TABLE 1
Expression Levels of Differentially Expressed Genes Involved in Actin Cytoskeleton
Signaling in Control NPCs and NPCs transfected with Gα₁₂ siRNA

| | | | Fold change (log₂ ratio) | |
|---|---|---|---|---|
| | | | Control_NPC- | Ga₁₂ siRNA_{—} |
| Symbol | GenBank ID | GeneName | TW06 | NPC-TW06 |
| AB12 | AI082434 | abl interactor 2 | −0.2396 | 0.6010 |
| ACTA2 | AI932231 | actin, alpha 2, smooth | −0.4176 | −1.0470 |
| | | muscle, aorta | | |
| ACTG2 | AA634006 | actin, gamma 2, smooth | | −0.8775 |
| | | muscle, enteric | | |
| ACTN1 | T60048 | actinin, alpha 1 | −1.3359 | −0.2620 |
| ACTN3 | AA669042 | actinin, alpha 3 | 0.8012 | 0.3466 |
| ACTN4 | AA196000 | actinin, alpha 4 | −0.0736 | 0.5338 |
| ALK | R66605 | anaplastic lymphoma kinase | | −0.7679 |
| | | (Ki-1) | | |
| APC | AA448482 | adenomatosis polyposis coli | 0.6006 | 0.5637 |
| ARHGEF12 | AA455997; | Rho guanine nucleotide | 3.1671 | −0.8820 |
| | AA410288 | exchange factor (GEF) 12 | | |
| ARHGEF7 | AA479287 | Rho guanine nucleotide | −1.4907 | −0.1536 |
| | | exchange factor (GEF) 7 | | |
| ARPC1B | AA490209 | actin related protein 2/3 | 1.6124 | 0.1881 |
| | | complex, subunit 1B, 41 kDa | | |
| ARPC5 | AA188179 | actin related protein 2/3 | 0.5385 | −1.4257 |
| | | complex, subunit 5, 16 kDa | | |
| BAIAP2 | W55964 | BAI1-associated protein 2 | | −1.0778 |
| BCAR1 | H46962 | breast cancer anti-estrogen | | −0.5743 |
| | | resistance 1 | | |
| CD14 | AA626335 | CD14 molecule | 0.5351 | −0.2849 |
| CFL2 | AA701476 | cofilin 2 (muscle) | 1.3997 | −0.4476 |
| DDR2 | AA598583 | discoidin domain receptor | 0.4418 | −0.5111 |
| (includes | | family, member 2 | | |
| EG: 4921) | | | | |
| DIAPH3 | AA620958 | diaphanous homolog 3 | −0.0620 | 0.4506 |
| | | (Drosophila) | | |
| DOCK1 | AI024983 | dedicator of cytokinesis 1 | 0.5189 | 0.7003 |
| EGFR | H11625; | epidermal growth factor | 0.4073 | −0.9580 |
| | AA001712 | receptor (erythroblastic leukemia | | |
| | | viral (v-erb-b) oncogene | | |
| | | homolog, avian) | | |
| F2R | H80439 | coagulation factor II (thrombin) | 0.3421 | −0.5362 |
| | | receptor | | |
| FGFR1 | AA400047 | fibroblast growth factor | −1.0682 | −0.1401 |
| | | receptor 1 (fms-related tyrosine | | |
| | | kinase 2, Pfeiffer syndrome) | | |
| FGFR3 | AA281064 | fibroblast growth factor | −1.4973 | −0.7884 |
| | | receptor 3 (achondroplasia, | | |
| | | thanatophoric dwarfism) | | |
| FGFR4 | AA419620 | fibroblast growth factor | −1.5952 | −0.1029 |
| | | receptor 4 | | |
| FN1 | AA446876; | fibronectin 1 | −4.7319 | −1.2984 |
| | AA127063 | | | |
| GNA12 | R62612; | guanine nucleotide binding | 1.2443 | −2.4673 |
| | H79130 | protein (G protein) alpha 12 | | |
| GPR161 | AI051410 | G protein-coupled receptor 161 | −1.0105 | −0.7984 |
| GRLF1 | R43550 | glucocorticoid receptor DNA | −4.5805 | |
| | | binding factor 1 | | |
| HRAS | AA489679 | v-Ha-ras Harvey rat sarcoma | 0.8172 | 0.1410 |
| | | viral oncogene homolog | | |
| IQGAP1 | AI536679; | IQ motif containing GTPase | 1.0675 | −0.7747 |
| (includes | AA757532 | activating protein 1 | | |
| EG: 8826) | | | | |
| IQGAP2 | AI285860 | IQ motif containing GTPase | 0.6005 | 0.3138 |
| | | activating protein 2 | | |
| ITGA2 | W32272 | integrin, alpha 2 (CD49B, alpha | | 0.5968 |
| | | 2 subunit of VLA-2 receptor) | | |
| ITGA3 | AA993294 | integrin, alpha 3 (antigen | −0.5662 | −0.2868 |
| | | CD49C, alpha 3 subunit of | | |
| | | VLA-3 receptor) | | |
| ITGA4 | AA424695 | integrin, alpha 4 (antigen | −1.3840 | |
| | | CD49D, alpha 4 subunit of | | |
| | | VLA-4 receptor) | | |
| ITGA6 | W73004 | integrin, alpha 6 | 0.9191 | |
| ITGAE | AW009867 | integrin, alpha E (antigen | 2.4453 | −0.0185 |
| | | CD103, human mucosal | | |
| | | lymphocyte antigen 1; alpha | | |
| | | polypeptide) | | |
| ITGAV | AA425451 | integrin, alpha V (vitronectin | −1.6403 | −0.5640 |
| | | receptor, alpha polypeptide, | | |
| | | antigen CD51) | | |
| ITGB6 | AA029934 | integrin, beta 6 | | 0.5440 |
| LTK | AA486731 | leukocyte tyrosine kinase | | −0.5772 |
| MAP2K1 | AI365420 | mitogen-activated protein | 0.0106 | −0.6741 |
| | | kinase kinase 1 | | |
| MAP2K2 | R44740 | mitogen-activated protein | −0.8102 | 0.3745 |
| | | kinase kinase 2 | | |
| MAPK1 | R11661 | mitogen-activated protein | −3.0472 | |
| | | kinase 1 | | |
| MYH11 | R22977 | myosin, heavy chain 11, | 0.5700 | −0.7197 |
| | | smooth muscle | | |
| MYH9 | AA488898 | myosin, heavy polypeptide 9, | −1.4465 | −0.4638 |
| | | non-muscle | | |
| MYL1 | T69926 | myosin, light chain 1, alkali; | −0.4643 | −0.8535 |
| | | skeletal, fast | | |
| MYL3 | AA196393 | myosin, light chain 3, alkali; | | −1.1948 |
| | | ventricular, skeletal, slow | | |
| MYL9 | AA192166 | myosin, light chain 9, | −0.6006 | −0.6488 |
| | | regulatory | | |
| MYLK | AI091881 | myosin, light chain kinase | −1.5056 | 0.7887 |
| NCKAP1 | AI972269 | NCK-associated protein 1 | | 0.6044 |
| NCKAP1L | AA099105 | NCK-associated protein 1-like | | −1.3788 |
| PAK1 | AA668726 | p21/Cdc42/Rac1-activated | 0.0592 | −1.0014 |
| | | kinase 1 (STE20 homolog, | | |
| | | yeast) | | |
| PAK2 | AA890663 | p21 (CDKN1A)-activated | 0.6660 | −0.5724 |
| | | kinase 2 | | |
| PAK3 | AI090533 | p21 (CDKN1A)-activated | 1.4301 | −0.7945 |
| | | kinase 3 | | |
| PAK6 | AI123354 | p21 (CDKN1A)-activated | −0.1136 | −1.1210 |
| | | kinase 6 | | |
| PDGFB | H15288; | platelet-derived growth factor | 0.4149 | 0.6905 |
| | W72000 | beta polypeptide (simian | | |
| | | sarcoma viral (v-sis) oncogene | | |
| | | homolog) | | |
| PDGFC | T49540 | platelet derived growth factor C | −0.1181 | −0.5394 |
| PDGFD | AA699775 | platelet derived growth factor D | −0.4072 | 0.6754 |
| PDGFRA | AI005125 | platelet-derived growth factor | 0.8634 | −0.4224 |
| | | receptor, alpha polypeptide | | |
| PFN1 | H23235 | profilin 1 | 0.4843 | 0.2496 |
| PIK3C3 | AA521431 | phosphoinositide-3-kinase, | | −1.6280 |
| | | class 3 | | |
| PIK3CA | R56397 | phosphoinositide-3-kinase, | 0.4628 | −0.7628 |
| | | catalytic, alpha polypeptide | | |
| PIK3CB | R22204 | phosphoinositide-3-kinase, | 0.4875 | −0.1104 |
| | | catalytic, beta polypeptide | | |
| PIK3CD | AA191461 | phosphoinositide-3-kinase, | 0.7904 | −0.1638 |
| | | catalytic, delta polypeptide | | |
| PIK3CG | AA186335 | phosphoinositide-3-kinase, | −2.0960 | −0.0678 |
| | | catalytic, gamma polypeptide | | |
| P1K3R1 | AA464176 | phosphoinositide-3-kinase, | 3.9177 | −1.1205 |
| | | regulatory subunit 1 (p85 alpha) | | |
| PIK3R3 | AA463460 | phosphoinositide-3-kinase, | | −0.8328 |
| | | regulatory subunit 3 (p55, | | |
| | | gamma) | | |
| PIK3R5 | AI208897 | phosphoinositide-3-kinase, | −0.5286 | −0.8664 |
| | | regulatory subunit 5, p101 | | |
| PIP5K1A | N53376 | phosphatidylinositol-4-phosphate | | −1.1981 |
| | | 5-kinase, type I, alpha | | |
| PIP5K2A | AI051874 | phosphatidylinositol-4-phosphate | −0.1194 | −0.8742 |
| | | 5-kinase, type II, alpha | | |
| PIP5K3 | H93068 | phosphatidylinositol-3-phosphate/ | | −0.6016 |
| (includes | | phosphatidylinositol 5-kinase, | | |
| EG: 200576) | | type III | | |
| PPP1CA | H01820 | protein phosphatase 1, catalytic | 0.9719 | −0.2969 |
| | | subunit, alpha isoform | | |
| PPP1CB | AA443982 | protein phosphatase 1, catalytic | −3.4365 | −0.9621 |
| | | subunit, beta isoform | | |
| PPP1CC | R26186 | protein phosphatase 1, catalytic | 0.9585 | −0.6088 |
| | | subunit, gamma isoform | | |
| PPP1R12A | AA129931; | protein phosphatase 1, | −0.3222 | −1.2867 |
| | N39074 | regulatory (inhibitor) subunit | | |
| | | 12A | | |
| PPP1R12B | AA487028 | protein phosphatase 1, | −1.3903 | −1.1979 |
| | | regulatory (inhibitor) subunit | | |
| | | 12B | | |
| PTK2 | AA704332 | PTK2 protein tyrosine kinase 2 | −0.9566 | −0.8771 |
| RAC2 | N51585 | ras-related C3 botulinum toxin | −0.9793 | 0.1634 |
| | | substrate 2 (rho family, small | | |
| | | GTP binding protein Rac2) | | |
| RAF1 | AI862818 | v-raf-1 murine leukemia viral | −0.2301 | −0.4394 |
| | | oncogene homolog 1 | | |
| RDX | N30713 | radixin | 1.0746 | −0.6353 |
| RHOA | AA479781 | ras homolog gene family, | −0.3162 | −0.5250 |
| | | member A | | |
| ROCK1 | AA676955; | Rho-associated, coiled-coil | 0.7478 | 0.6434 |
| | AI492217 | containing protein kinase 1 | | |
| ROR1 | T57805 | receptor tyrosine kinase-like | −0.0359 | −1.2896 |
| (includes | | orphan receptor 1 | | |
| EG: 4919) | | | | |
| RRAS2 | W02753 | related RAS viral (r-ras) | 0.6049 | −0.1467 |
| | | oncogene homolog 2 | | |
| SOS1 | R21416; | son of sevenless homolog 1 | 1.8710 | −2.3078 |
| | T54672 | (Drosophila) | | |
| SOS2 | H64325 | son of sevenless homolog 2 | 0.1693 | −0.9374 |
| | | (Drosophila) | | |
| SSH1 | AA708240 | slingshot homolog 1 | −1.0159 | −0.5643 |
| | | (Drosophila) | | |
| TIAM1 | N94357 | T-cell lymphoma invasion and | −0.6723 | −0.0052 |
| | | metastasis 1 | | |
| TMSB4Y | AW003835 | thymosin, beta 4, Y-linked | −0.3464 | −0.7448 |
| TTN | N50556 | titin | | 0.5621 |
| VAV2 | AA872006 | vav 2 oncogene | 0.8131 | −0.6760 |
| VAV3 | H54025 | vav 3 oncogene | 0.3415 | −1.0364 |
| VCL | H10045 | vinculin | −1.7004 | −0.1584 |
| VIL2 | AA486728 | villin 2 (ezrin) | 1.1790 | −0.6639 |
| WAS | AA411440 | Wiskott-Aldrich syndrome | −2.4614 | −0.0876 |
| | | (eczema-thrombocytopenia) | | |
| WASL | H61193 | Wiskott-Aldrich syndrome-like | −1.3729 | −3.9802 |

QRT-PCR was performed using a LightCycler PCR system and the FastStart DNA Master SYBR Green I Kit (Roche Applied Science) to verify the expression levels of ten genes, i.e., Gα₁₂, IQGAP1, IRS1, ARHGEF12, APRC5, c-MYC, WASK, FYN, JAK1, and MAP4, in control cells and in Gα₁₂ siRNA-expressed cells, using the primers listed in Table 2 below:

TABLE 2
Primers Used in QRT-PCR analysis

| | | | |
|---|---|---|---|
| Gene product | Unigene | Forward primer | Reverse primer |

| | | | |
|---|---|---|---|
| Guanine | Hs.487341 | GTTTGTCGTCGTTGAGC | AGTAGTTTCACTCGCCC |
| nucleotide-binding | | | |
| protein alpha-12 | | | |
| subunit (G alpha-12) | | | |
| IQ motif containing | Hs.430551 | CCCAAAGAACAAATACCAGG | GGCTAAGTTATCCAAGCAG |
| GTPase activating | | | |
| protein 1 (IQGAP1) | | | |
| Wiskott-Aldrich | Hs.143728 | ATTAGAGAGGGTGCTCAG | ATGAATGGCTTTGCTCC |
| syndrome-like; Neural | | | |
| Wiskott-Aldrich | | | |
| syndrome protein | | | |
| (N-WASP) | | | |
| Rho guanine | Hs.24598 | AGTTACACCATTCTTTGCC | GCACCTTGGGACTTGA |
| nucleotide exchange | | | |
| factor 12 | | | |
| (ARHGEF 12) | | | |
| v-myc | Hs.143728 | CATCAGCACAACTACGC | CTCGTTCCTCCTCTGG |
| myelocytomatosis viral | | | |
| oncogene homolog | | | |
| (avian v-MYC)) | | | |
| Actin-related protein | Hs.703792 | CTTGAAGGTGCTCATCT | GGACCCTACTCCTCCAG |
| 2/3 complex subunit 5 | | | |
| (ARP2/3 complex 16 | | | |
| kDa subunit) | | | |
| (p16-ARC; ARPC5) | | | |
| insulin receptor | Hs.471508 | GAAGACCTAAATGACCTCAG | TTTTCGCTTGGCACAAT |
| substrate 1 (IRS1) | | | |
| Janus kinase 1 (JAK1) | Hs.207538 | TGTAAGGGGATGGACTATTT | AACATTCTGGAGCATACC |
| FYN oncogene related | Hs.390567 | AGAGACAGGTTACATTCCC | TCCCAATCACGGATAGAAAG |
| to SRC, FGR, YES | | | |
| (FYN) | | | |
| ras homolog gene | Hs.247077 | TCGTTAGTCCACGGTCT | AACTGGTCCTTGCTGA |
| family, member A | | | |
| (RHOA) | | | |
| Microtubule-associated | Hs.517949 | AGCATTCAGTAGAGAAAGTC | GTCTTCCAGTAAGTCAGG |
| protein 4 (MAP4) | | | |

The gene expression levels obtained from the QRT-PCR assay were normalized against the expression level of MAP4. The results thus obtained were consistent with the microarray results shown in Table 1 above.

QRT-PCR was also performed to examine the expression levels of 8 epithelial-mesenchymal transition (EMT)-related genes, i.e., IQGAP1, ARHGEF12, JAK1, IRS1, ARPC5, v-MYC, RHOA, and WASL, in NPC-TW06 cells. All of these genes were found to be down-regulated in Gα₁₂ siRNA-expressed NPC cells. Overexpression of the wild-type Gα₁₂ and the Gα₁₂Q231L mutant increases the expression of IQGAP1 and RHOA. Western blot analysis was conducted to examine the protein levels of EMT markers in NPC-TW06 cells. Results thus obtained show that expression of LAMB3, vimentin, and paxillin were down-regulated by depletion of Gα₁₂ via RNA interference and up-regulated by overexpression of the wild-type Gα₁₂ and the Gα₁₂Q231L mutant. In addition, the protein levels of LAMB3, vimentin, and paxillin were down-regulated by depletion of IQGAP1, using a number of siRNAs targeting IQGAP1 (IQGAP1-siRNAs; see Example 4 below), and were up-regulated by IQGAP1 overexpression. In a wound-healing assay, IQGAP1-siRNAs significantly reduced the migration ability of NPC cells as compared to a control siRNA.

### Example 4

### Reduction of NPC Cancer Cell Mobility by Suppressing IQGAP1 Expression

CNE1 cells and NPC-TW06 cells were seeded at 5×10⁴ cells per well in 24-well plate. Twenty-four hours later, the cells were transfected with a number of IQGAP1-siRNAs (5′-GAACGUGGCUUAUGAGUACUU-3′,5′-GCAGGUGGAUUACUAUAAAUU-3′,5′-CGAACCAUCUUACUGAAUAUU-3′,5′-CAAUUGAGCAGUUCAGUUAUU-3′), or a control siRNA using the DharmaFECT 1 reagent (Dharmacon). The transfected cells were cultured for 1-3 days before subjected to the functional assays described below.

The mobility of the transfected cells was tested by the wound healing assay described in Example 3 above. 24 hours after transfection, the IQGAP1-siRNA-transfected NPC-TW06 cells showed markedly reduced mobility relative to the control-siRNA-transfected cells. This result indicates that suppression of IQGAP1 expression successfully reduced the migration ability of NPC cancer cells.

The siRNA transfected cells were then analyzed by immunostaining to examine the expression levels of vimentin and paxillin, both of which are markers of mesenchymal-like cells. Briefly, cells were fixed and immunostained using a mouse anti-vimentin antibody (Sigma) and a mouse anti-Paxillin (BD Transduction Laboratories). Results thus obtained show that the levels of both vimentin and paxillin were much lower in IQGAP1-siRNA transfected cells than those in control-siRNA transfected cells. Observed under a phase-contract microscope, the IQGAP1-siRNA transfected cells had an epithelioid-like appearance, i.e., flat and spread out, while the untrsfected cells had a fibroblastoid appearance, i.e., round and spindle-shaped. These results indicate that down-regulation of IQGAP1 expression results in morphology change of NPC cells in the same manner as that induced by down-regulation of Gα₁₂ expression.

### Other Embodiments

All of the features disclosed in this specification may be combined in any combination. Each feature disclosed in this specification may be replaced by an alternative feature serving the same, equivalent, or similar purpose. Thus, unless expressly stated otherwise, each feature disclosed is only an example of a generic series of equivalent or similar features.

From the above description, one skilled in the art can easily ascertain the essential characteristics of the present invention, and without departing from the spirit and scope thereof, can make various changes and modifications of the invention to adapt it to various usages and conditions. Thus, other embodiments are also within the claims.

TABLE
Significant GO categories affected in NPC

| | | Percent of | Permute P value | | | | | | |
|---|---|---|---|---|---|---|---|---|---|
| | GO term (Biological | genes present | T1-T2b | | | | T3-T4 | | |
| Group | process) | on chip* | NPC014 | NPC023 | NPC025 | NPC026 | NPC003 | NPC008 | NPC009 |
| I | negative regulation of cellular | 85.14 | 0.002 | 0.357 | 0.032 | 0.053 | 0.643 | 1 | 0.048 |
| | metabolism | | | | | | | | |
| | physiological process | 73.86 | 0.962 | 0 | 0.002 | 0.081 | 0.041 | 0 | 0 |
| | DNA packaging | 67.08 | 0.079 | 0.005 | 0 | 0.03 | 0.007 | 0.001 | 0.018 |
| | negative regulation of cellular | 85.03 | 0.091 | 0.72 | 0.006 | 0.031 | 0.059 | 0.135 | 0.032 |
| | physiological process | | | | | | | | |
| | organelle organization and | 77.16 | 0.224 | 0.004 | 0.101 | 0.004 | 0.001 | 0.002 | 0.018 |
| | biogenesis | | | | | | | | |
| | cellular physiological process | 76.78 | 0.236 | 0 | 0 | 0.014 | 0.005 | 0 | 0 |
| | neuropeptide signaling pathway | 65.33 | 0.001 | 0.094 | 0.176 | 0.037 | 0.084 | 0.139 | 0.302 |
| | chromosome organization and | 69.68 | 0.14 | 0.004 | 0.001 | 0.02 | 0.002 | 0.025 | 0.016 |
| | biogenesis | | | | | | | | |
| | chromosome organization and | 69.29 | 0.085 | 0.004 | 0.001 | 0.037 | 0.006 | 0.032 | 0.014 |
| | biogenesis (sensu Eukaryota) | | | | | | | | |
| | protein modification | 83.08 | 0.003 | 0 | 0 | 0.001 | 0 | 0.002 | 0.001 |
| | biopolymer modification | 83.17 | 0.003 | 0 | 0 | 0.001 | 0.001 | 0 | 0.001 |
| | transcription from RNA polymerase II | 88.19 | 0.047 | 0.001 | 0 | 0 | 0.002 | 0 | 0 |
| | promoter | | | | | | | | |
| | transcription | 75.51 | 0.008 | 0.445 | 0.002 | 0 | 0.112 | 0.001 | 0.031 |
| | regulation of metabolism | 75.77 | 0.02 | 0.141 | 0.016 | 0 | 0.165 | 0.003 | 0.008 |
| | transcription\, DNA-dependent | 75.09 | 0.012 | 0.388 | 0.007 | 0 | 0.071 | 0.004 | 0.041 |
| | regulation of transcription | 75.05 | 0.013 | 0.403 | 0.012 | 0 | 0.205 | 0.005 | 0.026 |
| | regulation of transcription\, DNA- | 74.76 | 0.018 | 0.429 | 0.013 | 0 | 0.182 | 0.013 | 0.042 |
| | dependent | | | | | | | | |
| | establishment and/or maintenance of | 65.95 | 0.121 | 0.002 | 0 | 0.009 | 0.008 | 0.015 | 0.037 |
| | chromatin architecture | | | | | | | | |
| | cellular process | 73.22 | 0.273 | 0 | 0.002 | 0.166 | 0.049 | 0.002 | 0.025 |
| | biological_process | 72.68 | 0.59 | 0.015 | 0.037 | 0.236 | 0.361 | 0.001 | 0.077 |
| | sodium ion transport | 78.89 | 1 | 0.033 | 1 | 0.001 | 0.025 | 0.008 | 0.024 |
| | regulation of nucleobase\, | 75.31 | 0.009 | 0.335 | 0.013 | 0 | 0.202 | 0.003 | 0.025 |
| | nucleoside\, nucleotide and nucleic | | | | | | | | |
| | acid metabolism | | | | | | | | |
| | regulation of cellular metabolism | 75.54 | 0.01 | 0.137 | 0.013 | 0 | 0.206 | 0.002 | 0.012 |
| | DNA metabolism | 69.75 | 0.139 | 0.013 | 0.129 | 0.039 | 0.099 | 0.035 | 0.266 |
| | RNA metabolism | 83.44 | 0.024 | 0.004 | 0.419 | 0.289 | 0.002 | 0 | 0.061 |
| | potassium ion transport | 75.00 | 0.008 | 0.019 | 0.043 | 0.305 | 0.002 | 0.335 | 0.291 |
| | RNA processing | 82.13 | 0.015 | 0.025 | 0.621 | 0.161 | 0 | 0.002 | 0.165 |
| | mRNA metabolism | 82.10 | 0.03 | 0.01 | 0.016 | 0.02 | 0 | 0 | 0.103 |
| | mRNA processing | 82.08 | 0.022 | 0.012 | 0.01 | 0.019 | 0 | 0 | 0.138 |
| | ion transport | 73.80 | 0.015 | 0.043 | 0.056 | 0.077 | 0.004 | 0.158 | 0.015 |
| | ubiquitin-dependent protein | 85.47 | 0.828 | 0.023 | 0.039 | 0.038 | 1 | 0.062 | 0.022 |
| | catabolism | | | | | | | | |
| | cation transport | 73.54 | 0.012 | 0.027 | 0.351 | 0.055 | 0 | 0.616 | 0.125 |
| | modification-dependent protein | 85.47 | 0.828 | 0.023 | 0.039 | 0.038 | 1 | 0.062 | 0.022 |
| | catabolism | | | | | | | | |
| | RNA splicing | 84.77 | 0.014 | 0.011 | 0.008 | 0.018 | 0 | 0 | 0.037 |
| | RNA splicing\, via transesterification | 83.43 | 0.051 | 0.01 | 0.011 | 0.015 | 0 | 0 | 0.079 |
| | reactions with bulged adenosine as | | | | | | | | |
| | nucleophile | | | | | | | | |
| | nuclear mRNA splicing\, via | 83.43 | 0.051 | 0.01 | 0.011 | 0.015 | 0 | 0 | 0.079 |
| | spliceosome | | | | | | | | |
| | metal ion transport | 73.73 | 0.035 | 0.008 | 0.268 | 0.017 | 0.001 | 0.177 | 0.009 |
| | RNA splicing\, via transesterification | 83.43 | 0.051 | 0.01 | 0.011 | 0.015 | 0 | 0 | 0.079 |
| | reactions | | | | | | | | |
| | protein metabolism | 76.16 | 0.285 | 0 | 0.01 | 0 | 0 | 0.001 | 0 |
| | cellular metabolism | 75.79 | 0.021 | 0 | 0 | 0 | 0 | 0 | 0 |
| | cellular protein metabolism | 76.14 | 0.284 | 0 | 0.007 | 0.001 | 0 | 0.001 | 0 |
| | primary metabolism | 75.82 | 0.014 | 0 | 0 | 0 | 0 | 0 | 0 |
| | metabolism | 75.79 | 0.031 | 0 | 0 | 0 | 0 | 0 | 0 |
| | cellular macromolecule metabolism | 76.36 | 0.501 | 0 | 0.01 | 0 | 0 | 0 | 0 |
| | macromolecule metabolism | 76.16 | 0.504 | 0 | 0.029 | 0.001 | 0 | 0 | 0 |
| | regulation of cellular physiological | 77.85 | 0.054 | 0.135 | 0.002 | 0.002 | 0.07 | 0.005 | 0.002 |
| | process | | | | | | | | |
| | regulation of biological process | 78.31 | 0.041 | 0.167 | 0.006 | 0.006 | 0.104 | 0.003 | 0.002 |
| | regulation of cellular process | 78.37 | 0.05 | 0.174 | 0.008 | 0.006 | 0.092 | 0.008 | 0.002 |
| | regulation of physiological process | 77.86 | 0.052 | 0.163 | 0.002 | 0.004 | 0.066 | 0.007 | 0.002 |
| | G-protein coupled receptor protein | 34.58 | 0.715 | 0.001 | 0.166 | 0 | 0 | 0 | 0.003 |
| | signaling pathway | | | | | | | | |
| | biopolymer metabolism | 78.67 | 0 | 0 | 0.001 | 0 | 0 | 0 | 0 |
| | ubiquitin cycle | 82.73 | 0.089 | 0.011 | 0.032 | 0.008 | 0.352 | 0.085 | 0 |
| | nucleobase\, nucleoside\, nucleotide | 75.04 | 0.002 | 0.007 | 0.003 | 0 | 0.008 | 0 | 0.002 |
| | and nucleic acid metabolism | | | | | | | | |
| II | macromolecule biosynthesis | 69.11 | 0.584 | 0.001 | 0.591 | 0.509 | 0.569 | 0.003 | 0.049 |
| | protein complex assembly | 68.18 | 0.784 | 0 | 1 | 0.34 | 0.02 | 0.046 | 0.214 |
| | cell organization and biogenesis | 78.21 | 0.175 | 0.004 | 0.096 | 0.064 | 0 | 0.002 | 0.016 |
| | protein polymerization | 65.85 | 0.426 | 0.018 | 0.237 | 0.839 | 0.06 | 0.03 | 0 |
| | cell division | 89.66 | 0.349 | 0.145 | 0.135 | 0.071 | 0.01 | 0.025 | 0.209 |
| | cytokinesis | 89.66 | 0.349 | 0.145 | 0.135 | 0.071 | 0.01 | 0.025 | 0.209 |
| | protein biosynthesis | 68.22 | 0.497 | 0.003 | 0.689 | 0.781 | 0.836 | 0.002 | 0.04 |
| | energy derivation by oxidation of | 82.52 | 0.065 | 0 | 0.929 | 0.168 | 0.08 | 0.009 | 0.038 |
| | organic compounds | | | | | | | | |
| | regulation of DNA metabolism | 96.43 | 0.217 | 0.049 | 0.141 | 0.861 | 0.303 | 0.158 | 0.31 |
| | cell proliferation | 83.88 | 0.5 | 0.872 | 0.036 | 0.59 | 0.105 | 0.019 | 0.041 |
| | cell cycle | 87.27 | 0.715 | 0.062 | 0.047 | 0.182 | 0.094 | 0.291 | 0.029 |
| | lipid catabolism | 70.59 | 0.586 | 0.028 | 0.254 | 0.316 | 0.412 | 0.248 | 0.096 |
| | regulation of cell cycle | 88.58 | 0.853 | 0.212 | 0.138 | 0.041 | 0.331 | 0.476 | 0.03 |
| | microtubule polymerization | 54.17 | 0.785 | 0.401 | 0.794 | 0.079 | 0.142 | 0.011 | 0.005 |
| III | heterocycle metabolism | 89.09 | 0.778 | 0.455 | 0.003 | 0.68 | 0.204 | 0.649 | 0.385 |
| | hexose catabolism | 73.53 | 0.049 | 0.026 | 0.138 | 0.197 | 0.288 | 0.072 | 0.027 |
| | transcriptional preinitiation complex | 83.33 | 0.039 | 0.365 | 1 | 0.205 | 0.351 | 0.182 | 0.013 |
| | formation | | | | | | | | |
| | hydrogen transport | 70.89 | 0.228 | 0.037 | 0.782 | 1 | 0.397 | 0.037 | 0.011 |
| | negative regulation of development | 83.78 | 0.122 | 0.045 | 0.845 | 0.009 | 0.45 | 0.026 | 0.449 |
| | monosaccharide catabolism | 72.46 | 0.049 | 0.026 | 0.138 | 0.197 | 0.288 | 0.072 | 0.027 |
| | oxidative phosphorylation | 64.84 | 0.013 | 0.031 | 0.26 | 1 | 0.595 | 0.013 | 0.013 |
| | cofactor metabolism | 81.40 | 0.046 | 0.045 | 0.157 | 0.858 | 1 | 0.151 | 0.012 |
| | response to bacteria | 59.49 | 0.039 | 0.178 | 0.025 | 0.002 | 0.368 | 0.064 | 1 |
| | alcohol catabolism | 72.46 | 0.049 | 0.026 | 0.138 | 0.197 | 0.288 | 0.072 | 0.027 |
| | main pathways of carbohydrate | 80.21 | 0.078 | 0 | 0.539 | 0.409 | 0.095 | 0.061 | 0.043 |
| | metabolism | | | | | | | | |
| | dephosphorylation | 81.82 | 0.005 | 0.002 | 0.045 | 0.044 | 0.077 | 0.12 | 0.027 |
| | cell-cell signaling | 69.41 | 0.004 | 0.68 | 0.245 | 0.001 | 0.03 | 0 | 0.37 |
| | protein amino acid | 80.95 | 0.009 | 0.001 | 0.105 | 0.039 | 0.068 | 0.058 | 0.009 |
| | dephosphorylation | | | | | | | | |
| | energy coupled proton transport\, | 67.35 | 0.109 | 0.004 | 0.859 | 0.509 | 0.719 | 0.011 | 0.014 |
| | down electrochemical gradient | | | | | | | | |
| | glycolysis | 71.43 | 0.073 | 0 | 0.484 | 0.032 | 0.014 | 0.063 | 0.052 |
| | ATP synthesis coupled proton | 67.35 | 0.109 | 0.004 | 0.859 | 0.509 | 0.719 | 0.011 | 0.014 |
| | transport | | | | | | | | |
| | morphogenesis | 79.87 | 0.764 | 0.574 | 0.033 | 0.101 | 0.007 | 0.367 | 0.566 |
| | phosphate metabolism | 82.74 | 0.003 | 0.003 | 0.005 | 0.042 | 0 | 0.007 | 0.003 |
| | phosphorus metabolism | 82.74 | 0.003 | 0.003 | 0.005 | 0.042 | 0 | 0.007 | 0.003 |
| | protein amino acid phosphorylation | 85.93 | 0.003 | 0.203 | 0.014 | 0.099 | 0.001 | 0.202 | 0.167 |
| | phosphorylation | 82.87 | 0.039 | 0.031 | 0.026 | 0.124 | 0.001 | 0.03 | 0.019 |
| | chromatin assembly or disassembly | 55.26 | 0.331 | 0.011 | 0.02 | 0.055 | 0.086 | 0.505 | 0.282 |
| | generation of precursor metabolites | 76.37 | 0.03 | 0.026 | 1 | 0.07 | 0.859 | 0.003 | 0.002 |
| | and energy | | | | | | | | |
| | chromatin modification | 91.00 | 0.382 | 0.072 | 0.011 | 0.291 | 0.071 | 0.047 | 0.187 |
| | cofactor biosynthesis | 76.11 | 0.379 | 0.033 | 0.109 | 1 | 0.444 | 0.009 | 0.002 |
| | glucose metabolism | 77.91 | 0.033 | 0.067 | 0.257 | 0.909 | 0.188 | 0.042 | 0.176 |
| | heme biosynthesis | 100.00 | 0.017 | 0.576 | 0.01 | 0.575 | 0.035 | 0.089 | 1 |
| | mRNA cleavage | 83.33 | 0.032 | 1 | 0.04 | 0.674 | 0.345 | 0.009 | 1 |
| | cellular biosynthesis | 73.29 | 0.58 | 0.001 | 0.935 | 0.944 | 0.422 | 0 | 0.089 |
| | negative regulation of transferase | 92.86 | 0.022 | 0.334 | 0.838 | 0.02 | 0.671 | 0.684 | 0.038 |
| | activity | | | | | | | | |
| | negative regulation of protein kinase | 92.86 | 0.022 | 0.334 | 0.838 | 0.02 | 0.671 | 0.684 | 0.038 |
| | activity | | | | | | | | |
| | glucose catabolism | 74.58 | 0.024 | 0.007 | 0.207 | 0.152 | 0.091 | 0.073 | 0.12 |
| | regulation of myogenesis | 83.33 | 0.34 | 0.39 | 0.003 | 0.03 | 0.047 | 0.402 | 0.082 |
| | negative regulation of myogenesis | 100.00 | 0.34 | 0.39 | 0.003 | 0.03 | 0.047 | 0.402 | 0.082 |
| | ATP metabolism | 68.97 | 0.416 | 0.002 | 1 | 0.674 | 0.874 | 0.025 | 0.003 |
| | regulation of transcription from RNA | 90.05 | 0.191 | 0 | 0.243 | 0.001 | 0.02 | 0.018 | 0.003 |
| | polymerase II promoter | | | | | | | | |
| | negative regulation of protein | 81.25 | 1 | 0.006 | 0.78 | 0.006 | 0.042 | 0.802 | 0.562 |
| | biosynthesis | | | | | | | | |
| | B cell differentiation | 60.00 | 0.046 | 0.486 | 0.038 | 0.019 | 1 | 0.317 | 0.312 |
| | actin polymerization and/or | 88.00 | 0.663 | 0.032 | 0.24 | 0.039 | 0.028 | 0.836 | 0.006 |
| | depolymerization | | | | | | | | |
| | nucleoside phosphate metabolism | 66.67 | 0.292 | 0.012 | 0.842 | 0.645 | 0.72 | 0.022 | 0.005 |
| | group transfer coenzyme metabolism | 70.83 | 0.764 | 0.039 | 0.557 | 0.407 | 0.897 | 0.017 | 0 |
| | activation of JNK activity | 90.00 | 0.154 | 0.159 | 0.458 | 0.019 | 0.708 | 0.032 | 0.291 |
| | defense response to bacteria | 51.56 | 0.038 | 0.284 | 0.035 | 0.019 | 0.276 | 0.389 | 0.698 |
| | ATP biosynthesis | 66.67 | 0.292 | 0.012 | 0.842 | 0.645 | 0.72 | 0.022 | 0.005 |
| | pigment biosynthesis | 100.00 | 0.062 | 0.525 | 0.015 | 0.126 | 0.04 | 0.019 | 0.083 |
| | protein localization | 88.69 | 0.752 | 0.076 | 0.542 | 0.768 | 0.358 | 0.013 | 0.016 |
| | establishment of protein localization | 88.44 | 0.785 | 0.111 | 0.63 | 0.805 | 0.357 | 0.007 | 0.018 |
| | muscle development | 81.69 | 0.562 | 0.461 | 0.069 | 0.148 | 0.034 | 0.646 | 0.005 |
| | proteoglycan metabolism | 57.14 | 0.765 | 0.572 | 0.499 | 1 | 0.557 | 0.046 | 0.036 |
| IV | DNA replication | 78.77 | 0.748 | 0.921 | 0.151 | 0.103 | 0.781 | 0.59 | 0.921 |
| | nuclear transport | 82.35 | 0.477 | 0.31 | 0.732 | 0.515 | 0.045 | 0.082 | 0.648 |
| | regulation of DNA replication | 100.00 | 0.314 | 0.067 | 0.172 | 0.547 | 0.484 | 0.748 | 0.053 |
| | DNA-dependent DNA replication | 79.17 | 0.316 | 0.586 | 0.374 | 0.129 | 0.891 | 0.406 | 0.494 |
| | establishment of RNA localization | 73.17 | 0.018 | 1 | 0.254 | 1 | 0.112 | 0.367 | 0.584 |
| | nuclear export | 77.78 | 0.053 | 0.49 | 0.224 | 0.516 | 0.043 | 0.097 | 0.601 |
| | nucleic acid transport | 73.17 | 0.018 | 1 | 0.254 | 1 | 0.112 | 0.367 | 0.584 |
| | microtubule-based process | 75.86 | 0.914 | 0.481 | 0.305 | 1 | 0.469 | 0.93 | 0.558 |
| | RNA-nucleus export | 75.00 | 0.018 | 1 | 0.254 | 1 | 0.112 | 0.367 | 0.584 |
| | RNA transport | 73.17 | 0.018 | 1 | 0.254 | 1 | 0.112 | 0.367 | 0.584 |
| | ribosome biogenesis and assembly | 81.48 | 0.517 | 0.651 | 0.015 | 0.119 | 0.522 | 0.093 | 0.895 |
| | RNA localization | 73.17 | 0.018 | 1 | 0.254 | 1 | 0.112 | 0.367 | 0.584 |
| | nucleocytoplasmic transport | 83.49 | 0.818 | 0.284 | 0.914 | 0.837 | 0.056 | 0.153 | 0.824 |
| | nucleobase\, nucleoside\, nucleotide | 72.92 | 0.051 | 1 | 0.408 | 0.861 | 0.192 | 0.311 | 0.612 |
| | and nucleic acid transport | | | | | | | | |
| | oligopeptide transport | 66.67 | 1 | 0.663 | 1 | 1 | 0.606 | 0.311 | 0.29 |
| | protein-nucleus export | 100.00 | 1 | 0.211 | 1 | 0.121 | 0.653 | 0.048 | 1 |
| | mRNA transport | 67.65 | 0.011 | 1 | 0.141 | 0.412 | 0.089 | 0.841 | 1 |
| | NLS-bearing substrate-nucleus | 91.67 | 0.787 | 0.759 | 0.368 | 0.756 | 0.048 | 0.195 | 0.064 |
| | import | | | | | | | | |
| | protein-nucleus import\, docking | 100.00 | 0.433 | 0.044 | 0.555 | 0.46 | 0.047 | 0.18 | 0.795 |
| | positive regulation of JNK cascade | 60.00 | 0.243 | 0.561 | 1 | 0.118 | 1 | 0.069 | 0.585 |
| | protein folding | 78.83 | 0.21 | 0.725 | 0.535 | 0.139 | 0.12 | 1 | 0.76 |
| | negative regulation of cellular | 85.31 | 0.18 | 0.703 | 0.017 | 0.027 | 0.183 | 0.184 | 0.121 |
| | process | | | | | | | | |
| | negative regulation of metabolism | 85.63 | 0.008 | 0.229 | 0.057 | 0.102 | 0.787 | 0.925 | 0.053 |
| | RNA modification | 92.19 | 1 | 0.001 | 0.525 | 0.688 | 0.282 | 0.048 | 0.142 |
| | pentose-phosphate shunt | 80.00 | 0.063 | 0.722 | 0.725 | 1 | 0.728 | 0.712 | 1 |
| | spliceosome assembly | 80.95 | 0.623 | 1 | 0.803 | 0.807 | 0.583 | 0.226 | 0.464 |
| | polysaccharide catabolism | 71.43 | 0.336 | 0.644 | 1 | 1 | 0.334 | 0.634 | 1 |
| | NADPH regeneration | 80.00 | 0.063 | 0.722 | 0.725 | 1 | 0.728 | 0.712 | 1 |
| | regulation of muscle contraction | 80.77 | 0.837 | 0.649 | 0.15 | 1 | 0.837 | 0.826 | 0.023 |
| | regulation of DNA recombination | 88.89 | 0.462 | 0.733 | 0.741 | 1 | 0.12 | 0.282 | 1 |
| | interphase | 91.03 | 1 | 0.915 | 0.207 | 0.263 | 0.633 | 0.271 | 0.402 |
| | N-acetylglucosamine metabolism | 63.64 | 0.274 | 1 | 0.735 | 0.116 | 0.086 | 0.537 | 0.728 |
| | glucan catabolism | 100.00 | 0.336 | 0.644 | 1 | 1 | 0.334 | 0.634 | 1 |
| | carbohydrate transport | 75.00 | 0.539 | 0.307 | 1 | 0.555 | 0.285 | 1 | 1 |
| | cellular polysaccharide catabolism | 71.43 | 0.336 | 0.644 | 1 | 1 | 0.334 | 0.634 | 1 |
| | cytoplasmic calcium ion homeostasis | 55.26 | 0.821 | 1 | 0.661 | 0.082 | 0.671 | 0.52 | 0.653 |
| | glycogen catabolism | 100.00 | 0.604 | 0.648 | 1 | 0.647 | 0.596 | 0.301 | 1 |
| | glucosamine metabolism | 66.67 | 0.472 | 0.73 | 1 | 0.234 | 0.046 | 0.376 | 1 |
| | myoblast differentiation | 100.00 | 0.345 | 1 | 1 | 0.68 | 0.351 | 0.637 | 0.638 |
| | glycogen metabolism | 92.86 | 0.818 | 0.145 | 0.292 | 0.452 | 0.679 | 0.021 | 0.096 |
| | histone deacetylation | 100.00 | 0.774 | 0.139 | 0.196 | 0.79 | 0.76 | 0.543 | 0.352 |
| | homeostasis | 78.83 | 0.234 | 0.099 | 1 | 0.04 | 0.914 | 0.779 | 0.536 |
| | interphase of mitotic cell cycle | 91.03 | 1 | 0.915 | 0.207 | 0.263 | 0.633 | 0.271 | 0.402 |
| | viral infectious cycle | 76.67 | 0.525 | 0.693 | 0.502 | 0.525 | 1 | 0.839 | 0.653 |
| | tRNA metabolism | 90.70 | 0.821 | 0.5 | 0.907 | 0.831 | 0.551 | 0.916 | 0.236 |
| | intracellular signaling cascade | 82.35 | 0.005 | 0.571 | 0.167 | 0.882 | 0.97 | 0.193 | 0.595 |
| | cell communication | 68.45 | 0.112 | 0.586 | 0.325 | 0.735 | 0.659 | 0.723 | 0.816 |
| | negative regulation of physiological | 84.33 | 0.113 | 0.845 | 0.01 | 0.086 | 0.078 | 0.224 | 0.067 |
| | process | | | | | | | | |
| | ion homeostasis | 76.11 | 0.244 | 0.333 | 0.811 | 0.04 | 0.651 | 0.237 | 0.588 |
| | microtubule polymerization or | 63.33 | 0.812 | 0.098 | 0.819 | 0.058 | 0.22 | 0.062 | 0.008 |
| | depolymerization | | | | | | | | |
| | transition metal ion transport | 76.19 | 0.269 | 1 | 0.321 | 0.29 | 1 | 0.099 | 1 |
| | mRNA-nucleus export | 69.70 | 0.011 | 1 | 0.141 | 0.412 | 0.089 | 0.841 | 1 |
| | negative regulation of biological | 84.21 | 0.072 | 0.785 | 0.024 | 0.028 | 0.215 | 0.286 | 0.08 |
| | process | | | | | | | | |
| | phosphoenolpyruvate-dependent | 60.00 | 1 | 1 | 1 | 1 | 1 | 0.605 | 0.568 |
| | sugar phosphotransferase system | | | | | | | | |
| | translation | 83.15 | 0.575 | 0 | 0.879 | 0.937 | 0.871 | 0.001 | 0.361 |
| | nucleotide-sugar metabolism | 100.00 | 0.207 | 0.563 | 0.12 | 0.088 | 0.374 | 0.595 | 0.548 |
| | cation homeostasis | 75.26 | 0.121 | 0.546 | 0.592 | 0.084 | 1 | 0.225 | 0.914 |
| | di-\, tri-valent inorganic cation | 72.41 | 0.11 | 0.379 | 0.89 | 0.116 | 0.901 | 0.192 | 0.91 |
| | homeostasis | | | | | | | | |
| | calcium ion homeostasis | 70.97 | 0.206 | 0.531 | 1 | 0.063 | 0.638 | 0.107 | 0.664 |
| | negative regulation of cell | 84.50 | 0.222 | 0.313 | 0.345 | 0.56 | 0.265 | 0.022 | 0.376 |
| | proliferation | | | | | | | | |
| | cell homeostasis | 76.32 | 0.048 | 0.189 | 1 | 0.124 | 0.914 | 0.53 | 0.818 |
| | signal transduction | 65.96 | 0.04 | 0.582 | 0.422 | 0.295 | 0.906 | 0.59 | 0.964 |
| | cell ion homeostasis | 74.76 | 0.067 | 0.353 | 0.785 | 0.097 | 1 | 0.294 | 0.829 |
| | metal ion homeostasis | 73.91 | 0.096 | 0.386 | 0.893 | 0.101 | 1 | 0.207 | 1 |
| | viral life cycle | 75.61 | 0.706 | 0.463 | 0.857 | 0.731 | 0.847 | 0.567 | 0.449 |
| | tRNA modification | 92.59 | 1 | 0.006 | 0.326 | 1 | 0.783 | 0.086 | 0.195 |
| | amino acid activation | 93.88 | 0.868 | 0.015 | 0.371 | 0.766 | 0.867 | 0.05 | 0.233 |
| | regulation of cell shape | 100.00 | 0.039 | 0.006 | 1 | 0.185 | 0.34 | 1 | 0.38 |
| | excretion | 79.49 | 0.867 | 0.286 | 0.504 | 0.037 | 0.192 | 1 | 0.371 |
| | traversing start control point of | 100.00 | 1 | 0.662 | 1 | 0.688 | 1 | 0.428 | 1 |
| | mitotic cell cycle | | | | | | | | |
| | histidine catabolism | 100.00 | 0.55 | 1 | 0.25 | 0.281 | 1 | 0.559 | 0.292 |
| | NADP metabolism | 81.82 | 0.074 | 1 | 0.716 | 1 | 0.514 | 1 | 0.709 |
| | histidine family amino acid | 100.00 | 0.55 | 1 | 0.25 | 0.281 | 1 | 0.559 | 0.292 |
| | catabolism | | | | | | | | |
| | protein-nucleus import | 84.38 | 0.552 | 0.487 | 0.753 | 0.886 | 0.352 | 0.485 | 1 |
| | purine base metabolism | 100.00 | 0.182 | 0.367 | 1 | 0.671 | 0.343 | 0.641 | 0.628 |
| | negative regulation of cell cycle | 87.91 | 0.715 | 0.648 | 0.448 | 0.105 | 0.402 | 0.198 | 0.792 |
| | response to DNA damage stimulus | 81.07 | 0.937 | 0.048 | 0.608 | 1 | 0.532 | 0.133 | 0.763 |
| | tRNA aminoacylation | 93.88 | 0.868 | 0.015 | 0.371 | 0.766 | 0.867 | 0.05 | 0.233 |
| | negative regulation of protein | 88.89 | 0.733 | 0.006 | 1 | 0.224 | 0.066 | 0.585 | 0.2 |
| | metabolism | | | | | | | | |
| | sphingolipid biosynthesis | 76.92 | 1 | 0.053 | 0.735 | 0.545 | 0.542 | 0.092 | 0.189 |
| | response to endogenous stimulus | 80.82 | 1 | 0.06 | 1 | 0.896 | 0.312 | 0.084 | 0.698 |
| | tRNA aminoacylation for protein | 93.88 | 0.868 | 0.015 | 0.371 | 0.766 | 0.867 | 0.05 | 0.233 |
| | translation | | | | | | | | |
| | regulation of angiogenesis | 85.71 | 0.75 | 0.07 | 0.533 | 0.245 | 0.758 | 0.085 | 0.759 |
| | nuclear import | 84.38 | 0.552 | 0.487 | 0.753 | 0.886 | 0.352 | 0.485 | 1 |
| | phosphoinositide biosynthesis | 84.62 | 0.05 | 0.547 | 0.765 | 0.375 | 0.749 | 0.504 | 0.768 |
| | DNA repair | 81.28 | 0.877 | 0.092 | 0.462 | 0.94 | 0.501 | 0.283 | 0.886 |
| | intracellular transport | 84.01 | 0.545 | 0.059 | 0.087 | 0.959 | 0.013 | 0.003 | 0.237 |
| | cell surface receptor linked signal | 49.13 | 0.628 | 0.029 | 0.363 | 0.194 | 0.014 | 0.22 | 0.77 |
| | transduction | | | | | | | | |
| | vasodilation | 100.00 | 0.249 | 0.068 | 0.245 | 0.134 | 0.575 | 0.069 | 1 |
| | cytoskeleton organization and | 81.00 | 0.589 | 0.065 | 0.396 | 0.009 | 0.177 | 0.014 | 0.06 |
| | biogenesis | | | | | | | | |
| | monovalent inorganic cation | 73.97 | 0.027 | 0.222 | 0.118 | 0.088 | 0 | 0.444 | 0.777 |
| | transport | | | | | | | | |
| | complement activation\, classical | 75.00 | 0.819 | 1 | 0.266 | 1 | 0.385 | 0.253 | 0.515 |
| | pathway | | | | | | | | |
| | glycosphingolipid metabolism | 71.43 | 0.518 | 0.554 | 1 | 0.741 | 0.52 | 0.745 | 0.515 |
| | protein targeting | 85.71 | 0.621 | 0.061 | 0.462 | 0.522 | 0.458 | 0.051 | 0.306 |
| | humoral immune response | 78.13 | 0.622 | 0.457 | 0.033 | 0.484 | 0.11 | 0.261 | 0.058 |
| | regulation of cell proliferation | 80.95 | 0.399 | 0.53 | 0.15 | 0.463 | 0.132 | 0.085 | 0.232 |
| | regulation of vasodilation | 100.00 | 0.249 | 0.068 | 0.245 | 0.134 | 0.575 | 0.069 | 1 |
| | humoral defense mechanism (sensu | 76.07 | 0.478 | 0.52 | 0.022 | 0.519 | 0.263 | 0.466 | 0.16 |
| | Vertebrata) | | | | | | | | |
| | mitotic cell cycle | 92.67 | 0.243 | 0.212 | 0.465 | 0.751 | 0.697 | 0.396 | 0.474 |
| | translational elongation | 53.13 | 0.311 | 0.321 | 0.072 | 0.457 | 0.786 | 0.005 | 0.629 |
| | activation of MAPKK activity | 100.00 | 0.034 | 1 | 0.557 | 0.604 | 0.537 | 0.084 | 1 |

| | | Permute P value | | | | | | |
|---|---|---|---|---|---|---|---|---|
| | GO term (Biological | T3-T4 | | NPC-derived cell lines | | | | |
| Group | process) | NPC010 | NPC015 | CNE1 | CNE2 | HONE1 | NPC-TW01 | NPC-TW06 |
| I | negative regulation of cellular | 0.117 | 0.446 | 0.167 | 0.183 | 0.488 | 0.039 | 0.014 |
| | metabolism | | | | | | | |
| | physiological process | 0 | 0.12 | 0.001 | 0 | 0.001 | 0.004 | 0.002 |
| | DNA packaging | 0 | 0.348 | 0.017 | 0.06 | 0.004 | 0.009 | 0.059 |
| | negative regulation of cellular | 0.07 | 0.03 | 0.026 | 0.143 | 0.019 | 0.009 | 0.001 |
| | physiological process | | | | | | | |
| | organelle organization and | 0 | 0.075 | 0 | 0 | 0 | 0 | 0.022 |
| | biogenesis | | | | | | | |
| | cellular physiological process | 0 | 0.009 | 0 | 0 | 0 | 0 | 0 |
| | neuropeptide signaling pathway | 0.641 | 0.045 | 0.072 | 0.003 | 0.033 | 0.072 | 0.041 |
| | chromosome organization and | 0 | 0.194 | 0.011 | 0.012 | 0.002 | 0.005 | 0.037 |
| | biogenesis | | | | | | | |
| | chromosome organization and | 0 | 0.242 | 0.02 | 0.027 | 0.002 | 0.006 | 0.056 |
| | biogenesis (sensu Eukaryota) | | | | | | | |
| | protein modification | 0 | 0.029 | 0.119 | 0 | 0.044 | 0.057 | 0.155 |
| | biopolymer modification | 0 | 0.022 | 0.044 | 0 | 0.007 | 0.013 | 0.056 |
| | transcription from RNA polymerase II | 0.134 | 0.003 | 0.069 | 0.1 | 0.043 | 0.197 | 0.003 |
| | promoter | | | | | | | |
| | transcription | 0.73 | 0.047 | 0.054 | 0.01 | 0.191 | 0.339 | 0.033 |
| | regulation of metabolism | 0.606 | 0.02 | 0.027 | 0.016 | 0.086 | 0.122 | 0.006 |
| | transcription\, DNA-dependent | 0.667 | 0.03 | 0.059 | 0.019 | 0.298 | 0.391 | 0.032 |
| | regulation of transcription | 0.733 | 0.062 | 0.071 | 0.019 | 0.269 | 0.254 | 0.03 |
| | regulation of transcription\, DNA- | 0.709 | 0.041 | 0.077 | 0.035 | 0.516 | 0.445 | 0.038 |
| | dependent | | | | | | | |
| | establishment and/or maintenance of | 0 | 0.546 | 0.019 | 0.104 | 0.012 | 0.017 | 0.061 |
| | chromatin architecture | | | | | | | |
| | cellular process | 0 | 0.086 | 0.018 | 0.003 | 0.074 | 0.015 | 0.047 |
| | biological_process | 0 | 0.349 | 0.01 | 0.001 | 0.128 | 0.029 | 0.01 |
| | sodium ion transport | 0.248 | 0.012 | 0.028 | 0.158 | 0.038 | 0.161 | 0.08 |
| | regulation of nucleobase\, | 0.527 | 0.019 | 0.044 | 0.012 | 0.178 | 0.111 | 0.01 |
| | nucleoside\, nucleotide and nucleic | | | | | | | |
| | acid metabolism | | | | | | | |
| | regulation of cellular metabolism | 0.454 | 0.019 | 0.038 | 0.012 | 0.13 | 0.136 | 0.01 |
| | DNA metabolism | 0.028 | 0.335 | 0 | 0 | 0 | 0 | 0 |
| | RNA metabolism | 0.001 | 0.001 | 0 | 0 | 0 | 0 | 0 |
| | potassium ion transport | 0.086 | 0.546 | 0.004 | 0.002 | 0.002 | 0.004 | 0 |
| | RNA processing | 0.002 | 0 | 0 | 0 | 0 | 0.003 | 0 |
| | mRNA metabolism | 0 | 0 | 0 | 0 | 0 | 0.01 | 0 |
| | mRNA processing | 0.006 | 0 | 0 | 0 | 0 | 0.018 | 0 |
| | ion transport | 1 | 0.128 | 0.001 | 0.001 | 0 | 0.005 | 0 |
| | ubiquitin-dependent protein | 0.48 | 0 | 0 | 0.005 | 0 | 0.001 | 0.002 |
| | catabolism | | | | | | | |
| | cation transport | 0.444 | 0.227 | 0.002 | 0.006 | 0 | 0.008 | 0.001 |
| | modification-dependent protein | 0.48 | 0 | 0 | 0.005 | 0 | 0.001 | 0.002 |
| | catabolism | | | | | | | |
| | RNA splicing | 0.017 | 0 | 0 | 0 | 0 | 0.019 | 0.005 |
| | RNA splicing\, via transesterification | 0.017 | 0 | 0 | 0 | 0 | 0.008 | 0.003 |
| | reactions with bulged adenosine as | | | | | | | |
| | nucleophile | | | | | | | |
| | nuclear mRNA splicing\, via | 0.017 | 0 | 0 | 0 | 0 | 0.008 | 0.003 |
| | spliceosome | | | | | | | |
| | metal ion transport | 0.048 | 0.131 | 0.002 | 0.001 | 0 | 0.003 | 0.001 |
| | RNA splicing\, via transesterification | 0.017 | 0 | 0 | 0 | 0 | 0.008 | 0.003 |
| | reactions | | | | | | | |
| | protein metabolism | 0 | 0.09 | 0.003 | 0 | 0 | 0 | 0 |
| | cellular metabolism | 0 | 0 | 0 | 0 | 0 | 0 | 0 |
| | cellular protein metabolism | 0 | 0.07 | 0.003 | 0 | 0 | 0 | 0 |
| | primary metabolism | 0 | 0 | 0 | 0 | 0 | 0 | 0 |
| | metabolism | 0 | 0.006 | 0 | 0 | 0 | 0 | 0 |
| | cellular macromolecule metabolism | 0 | 0.018 | 0.001 | 0 | 0 | 0 | 0.001 |
| | macromolecule metabolism | 0 | 0.062 | 0 | 0 | 0 | 0 | 0.001 |
| | regulation of cellular physiological | 0.677 | 0.009 | 0.008 | 0.003 | 0.001 | 0.019 | 0 |
| | process | | | | | | | |
| | regulation of biological process | 0.617 | 0.041 | 0.006 | 0 | 0 | 0.012 | 0 |
| | regulation of cellular process | 0.883 | 0.02 | 0.016 | 0.001 | 0.002 | 0.041 | 0 |
| | regulation of physiological process | 0.712 | 0.02 | 0.008 | 0.001 | 0 | 0.008 | 0 |
| | G-protein coupled receptor protein | 0.062 | 0.046 | 0 | 0 | 0.005 | 0.003 | 0 |
| | signaling pathway | | | | | | | |
| | biopolymer metabolism | 0 | 0 | 0 | 0 | 0 | 0 | 0 |
| | ubiquitin cycle | 0.1 | 0.001 | 0.113 | 0.005 | 0.024 | 0.379 | 0.013 |
| | nucleobase\, nucleoside\, nucleotide | 0.16 | 0.001 | 0 | 0 | 0 | 0 | 0 |
| | and nucleic acid metabolism | | | | | | | |
| II | macromolecule biosynthesis | 0.218 | 1 | 0.195 | 0.063 | 0 | 0.066 | 0.006 |
| | protein complex assembly | 0.142 | 0.8 | 0.21 | 0.362 | 0.006 | 0.051 | 0.024 |
| | cell organization and biogenesis | 0 | 0.319 | 0 | 0 | 0 | 0.001 | 0.011 |
| | protein polymerization | 0.181 | 0.01 | 0.073 | 0.15 | 0.02 | 0.023 | 0.054 |
| | cell division | 0.011 | 0.015 | 0.009 | 0.024 | 0.004 | 0.014 | 0.01 |
| | cytokinesis | 0.011 | 0.015 | 0.009 | 0.024 | 0.004 | 0.014 | 0.01 |
| | protein biosynthesis | 0.263 | 0.962 | 0.264 | 0.018 | 0 | 0.037 | 0.002 |
| | energy derivation by oxidation of | 0.696 | 0.013 | 0.026 | 0.103 | 0.01 | 0.026 | 0.07 |
| | organic compounds | | | | | | | |
| | regulation of DNA metabolism | 0.009 | 0.019 | 0.026 | 0.033 | 0.009 | 0.002 | 0.007 |
| | cell proliferation | 0.058 | 0.044 | 0.115 | 0.03 | 0 | 0.001 | 0.021 |
| | cell cycle | 0.241 | 0.016 | 0.001 | 0.003 | 0 | 0 | 0 |
| | lipid catabolism | 0.038 | 0.047 | 0.016 | 0.003 | 0.031 | 0.013 | 0.031 |
| | regulation of cell cycle | 0.511 | 0.02 | 0 | 0.008 | 0 | 0 | 0 |
| | microtubule polymerization | 0.274 | 0.027 | 0.017 | 0.067 | 0.002 | 0.004 | 0.003 |
| III | heterocycle metabolism | 0.041 | 0.007 | 1 | 0.894 | 0.873 | 0.884 | 0.459 |
| | hexose catabolism | 0.08 | 0.086 | 0.565 | 0.684 | 0.037 | 0.279 | 0.362 |
| | transcriptional preinitiation complex | 0.014 | 1 | 0.678 | 1 | 0.649 | 1 | 0.35 |
| | formation | | | | | | | |
| | hydrogen transport | 0.294 | 0.91 | 0.468 | 1 | 0.684 | 0.792 | 0.68 |
| | negative regulation of development | 1 | 0.601 | 0.857 | 1 | 0.696 | 0.863 | 0.466 |
| | monosaccharide catabolism | 0.08 | 0.086 | 0.565 | 0.684 | 0.037 | 0.279 | 0.362 |
| | oxidative phosphorylation | 0.875 | 1 | 0.308 | 0.483 | 0.499 | 0.576 | 0.315 |
| | cofactor metabolism | 0.1 | 0.541 | 0.724 | 0.713 | 1 | 0.841 | 0.817 |
| | response to bacteria | 0.012 | 0.007 | 0.164 | 0.131 | 0.202 | 0.012 | 0.087 |
| | alcohol catabolism | 0.08 | 0.086 | 0.565 | 0.684 | 0.037 | 0.279 | 0.362 |
| | main pathways of carbohydrate | 0.729 | 0.029 | 0.143 | 0.496 | 0.032 | 0.309 | 0.283 |
| | metabolism | | | | | | | |
| | dephosphorylation | 0.051 | 0.625 | 0.119 | 0.005 | 0.064 | 0.258 | 0.756 |
| | cell-cell signaling | 0.053 | 0.035 | 0.057 | 0.016 | 0.056 | 0.119 | 0.072 |
| | protein amino acid | 0.036 | 0.423 | 0.119 | 0.005 | 0.058 | 0.203 | 0.611 |
| | dephosphorylation | | | | | | | |
| | energy coupled proton transport\, | 1 | 0.853 | 0.466 | 0.86 | 0.58 | 0.708 | 0.866 |
| | down electrochemical gradient | | | | | | | |
| | glycolysis | 0.218 | 0.038 | 0.469 | 0.881 | 0.027 | 0.132 | 0.22 |
| | ATP synthesis coupled proton | 1 | 0.853 | 0.466 | 0.86 | 0.58 | 0.708 | 0.866 |
| | transport | | | | | | | |
| | morphogenesis | 0.035 | 1 | 0.246 | 0.408 | 0.668 | 0.469 | 0.16 |
| | phosphate metabolism | 0.004 | 0.82 | 0.69 | 0.113 | 0.478 | 0.939 | 0.298 |
| | phosphorus metabolism | 0.004 | 0.82 | 0.69 | 0.113 | 0.478 | 0.939 | 0.298 |
| | protein amino acid phosphorylation | 0.005 | 1 | 0.583 | 0.58 | 1 | 0.917 | 0.327 |
| | phosphorylation | 0.007 | 0.897 | 0.398 | 0.53 | 1 | 0.876 | 0.248 |
| | chromatin assembly or disassembly | 0 | 0.642 | 0.577 | 0.834 | 0.317 | 0.33 | 0.662 |
| | generation of precursor metabolites | 0.435 | 0.044 | 0.556 | 1 | 0.647 | 0.777 | 0.74 |
| | and energy | | | | | | | |
| | chromatin modification | 0.004 | 0.66 | 0.101 | 0.174 | 0.389 | 0.308 | 0.332 |
| | cofactor biosynthesis | 0.159 | 0.828 | 0.424 | 0.83 | 0.498 | 0.733 | 0.679 |
| | glucose metabolism | 0.009 | 0.094 | 0.692 | 0.909 | 0.057 | 0.28 | 0.801 |
| | heme biosynthesis | 0.14 | 0.212 | 1 | 1 | 0.754 | 0.762 | 1 |
| | mRNA cleavage | 0.65 | 1 | 1 | 0.329 | 0.064 | 1 | 0.37 |
| | cellular biosynthesis | 0.049 | 0.72 | 0.618 | 0.154 | 0.022 | 0.514 | 0.127 |
| | negative regulation of transferase | 0.67 | 0.856 | 0.401 | 0.159 | 0.019 | 0.824 | 0.3 |
| | activity | | | | | | | |
| | negative regulation of protein kinase | 0.67 | 0.856 | 0.401 | 0.159 | 0.019 | 0.824 | 0.3 |
| | activity | | | | | | | |
| | glucose catabolism | 0.037 | 0.107 | 0.335 | 0.298 | 0.009 | 0.172 | 0.184 |
| | regulation of myogenesis | 0.65 | 1 | 0.339 | 1 | 0.375 | 0.694 | 0.324 |
| | negative regulation of myogenesis | 0.65 | 1 | 0.339 | 1 | 0.375 | 0.694 | 0.324 |
| | ATP metabolism | 0.532 | 0.73 | 0.487 | 0.741 | 1 | 1 | 1 |
| | regulation of transcription from RNA | 0.226 | 0.143 | 0.313 | 0.821 | 0.159 | 0.887 | 0.03 |
| | polymerase II promoter | | | | | | | |
| | negative regulation of protein | 0.256 | 1 | 0.765 | 0.261 | 0.547 | 0.386 | 0.243 |
| | biosynthesis | | | | | | | |
| | B cell differentiation | 0.194 | 0.292 | 0.721 | 0.726 | 0.723 | 0.15 | 0.301 |
| | actin polymerization and/or | 1 | 0.27 | 0.272 | 0.164 | 0.38 | 0.364 | 0.661 |
| | depolymerization | | | | | | | |
| | nucleoside phosphate metabolism | 0.872 | 0.582 | 0.271 | 1 | 0.607 | 0.721 | 0.879 |
| | group transfer coenzyme metabolism | 0.406 | 0.681 | 0.377 | 0.779 | 0.664 | 0.889 | 1 |
| | activation of JNK activity | 0.03 | 0.299 | 1 | 1 | 1 | 0.75 | 0.738 |
| | defense response to bacteria | 0.023 | 0.006 | 0.133 | 0.268 | 0.205 | 0.054 | 0.096 |
| | ATP biosynthesis | 0.872 | 0.582 | 0.271 | 1 | 0.607 | 0.721 | 0.879 |
| | pigment biosynthesis | 0.03 | 0.174 | 1 | 1 | 0.8 | 1 | 1 |
| | protein localization | 0.015 | 0.171 | 0.09 | 0.832 | 0.557 | 0.163 | 0.138 |
| | establishment of protein localization | 0.017 | 0.221 | 0.073 | 0.831 | 0.523 | 0.145 | 0.156 |
| | muscle development | 0.023 | 0.644 | 0.331 | 0.064 | 0.855 | 0.214 | 0.058 |
| | proteoglycan metabolism | 0.409 | 0.035 | 1 | 0.563 | 0.567 | 0.751 | 0.568 |
| IV | DNA replication | 0.837 | 0.365 | 0 | 0 | 0 | 0 | 0 |
| | nuclear transport | 0.843 | 1 | 0 | 0 | 0.01 | 0 | 0 |
| | regulation of DNA replication | 0.017 | 0.169 | 0.022 | 0.028 | 0.022 | 0.006 | 0.004 |
| | DNA-dependent DNA replication | 0.022 | 0.224 | 0.017 | 0.003 | 0 | 0.002 | 0 |
| | establishment of RNA localization | 1 | 1 | 0.044 | 0.008 | 0.094 | 0.001 | 0.066 |
| | nuclear export | 0.539 | 1 | 0.007 | 0.007 | 0.023 | 0.001 | 0.067 |
| | nucleic acid transport | 1 | 1 | 0.044 | 0.008 | 0.094 | 0.001 | 0.066 |
| | microtubule-based process | 0.919 | 0.674 | 0.022 | 0.132 | 0.171 | 0.076 | 0.047 |
| | RNA-nucleus export | 1 | 1 | 0.044 | 0.008 | 0.094 | 0.001 | 0.066 |
| | RNA transport | 1 | 1 | 0.044 | 0.008 | 0.094 | 0.001 | 0.066 |
| | ribosome biogenesis and assembly | 0.75 | 0.455 | 0.195 | 0.006 | 0.042 | 0.515 | 0.522 |
| | RNA localization | 1 | 1 | 0.044 | 0.008 | 0.094 | 0.001 | 0.066 |
| | nucleocytoplasmic transport | 0.856 | 1 | 0.002 | 0 | 0.015 | 0 | 0 |
| | nucleobase\, nucleoside\, nucleotide | 0.75 | 0.75 | 0.036 | 0.004 | 0.048 | 0 | 0.03 |
| | and nucleic acid transport | | | | | | | |
| | oligopeptide transport | 0.031 | 0.186 | 0.014 | 0.014 | 0.015 | 0.009 | 0.023 |
| | protein-nucleus export | 0.445 | 0.684 | 0.024 | 0.667 | 0.022 | 0.072 | 0.672 |
| | mRNA transport | 0.419 | 0.706 | 0.039 | 0.018 | 0.268 | 0.016 | 0.294 |
| | NLS-bearing substrate-nucleus | 0.366 | 0.118 | 0.048 | 0.048 | 0.059 | 0.115 | 0 |
| | import | | | | | | | |
| | protein-nucleus import\, docking | 1 | 0.291 | 0.054 | 0.184 | 0.017 | 0.029 | 0.176 |
| | positive regulation of JNK cascade | 0.578 | 1 | 0.037 | 0.034 | 0.046 | 0.026 | 0.047 |
| | protein folding | 0.605 | 0.507 | 0.012 | 0.047 | 0.033 | 0.002 | 0.017 |
| | negative regulation of cellular | 0.208 | 0.053 | 0.111 | 0.293 | 0.061 | 0.019 | 0.003 |
| | process | | | | | | | |
| | negative regulation of metabolism | 0.295 | 0.414 | 0.115 | 0.148 | 0.456 | 0.032 | 0.002 |
| | RNA modification | 0.182 | 0.216 | 0.053 | 0.005 | 0.004 | 0.06 | 0.056 |
| | pentose-phosphate shunt | 0.479 | 1 | 0.12 | 0.025 | 0.025 | 0.251 | 0.119 |
| | spliceosome assembly | 0.795 | 0.432 | 0.14 | 0.038 | 0.041 | 0.2 | 0.062 |
| | polysaccharide catabolism | 0.618 | 0.63 | 0.043 | 0.332 | 0.05 | 0.336 | 0.343 |
| | NADPH regeneration | 0.479 | 1 | 0.12 | 0.025 | 0.025 | 0.251 | 0.119 |
| | regulation of muscle contraction | 0.193 | 0.822 | 1 | 0.043 | 0.031 | 0.487 | 1 |
| | regulation of DNA recombination | 0.723 | 0.271 | 0.44 | 0.13 | 0.021 | 0.014 | 0.163 |
| | interphase | 0.726 | 0.098 | 0.072 | 0.104 | 0.05 | 0.021 | 0.099 |
| | N-acetylglucosamine metabolism | 0.527 | 1 | 0.019 | 0.024 | 0.304 | 0.273 | 0.33 |
| | glucan catabolism | 0.618 | 0.63 | 0.043 | 0.332 | 0.05 | 0.336 | 0.343 |
| | carbohydrate transport | 0.843 | 0.85 | 0.048 | 0.061 | 0.833 | 0.012 | 0.52 |
| | cellular polysaccharide catabolism | 0.618 | 0.63 | 0.043 | 0.332 | 0.05 | 0.336 | 0.343 |
| | cytoplasmic calcium ion homeostasis | 0.502 | 1 | 0.021 | 0.015 | 0.182 | 0.343 | 0.054 |
| | glycogen catabolism | 1 | 0.676 | 0.011 | 0.123 | 0.019 | 0.086 | 0.119 |
| | glucosamine metabolism | 0.359 | 0.737 | 0.006 | 0.009 | 0.505 | 0.333 | 0.21 |
| | myoblast differentiation | 1 | 1 | 0.368 | 0.335 | 0.049 | 0.032 | 0.352 |
| | glycogen metabolism | 0.027 | 0.434 | 0.038 | 0.053 | 0.135 | 0.021 | 0.067 |
| | histone deacetylation | 1 | 0.35 | 0.034 | 0.057 | 0.067 | 0.11 | 0.008 |
| | homeostasis | 0.629 | 0.843 | 0.01 | 0.014 | 0.117 | 0.167 | 0.113 |
| | interphase of mitotic cell cycle | 0.726 | 0.098 | 0.072 | 0.104 | 0.05 | 0.021 | 0.099 |
| | viral infectious cycle | 0.676 | 0.657 | 0.2 | 0.049 | 0.043 | 0.271 | 0.058 |
| | tRNA metabolism | 0.927 | 0.917 | 0.098 | 0.007 | 0.004 | 0.074 | 0.352 |
| | intracellular signaling cascade | 0.841 | 0.65 | 0.025 | 0.623 | 0.014 | 0.013 | 0.061 |
| | cell communication | 0.681 | 0.975 | 0.026 | 0.125 | 0.01 | 0.014 | 0.004 |
| | negative regulation of physiological | 0.1 | 0.067 | 0.065 | 0.204 | 0.047 | 0.022 | 0.002 |
| | process | | | | | | | |
| | ion homeostasis | 0.649 | 0.911 | 0.011 | 0.017 | 0.174 | 0.05 | 0.122 |
| | microtubule polymerization or | 0.823 | 0.038 | 0.009 | 0.132 | 0.01 | 0.021 | 0 |
| | depolymerization | | | | | | | |
| | transition metal ion transport | 0.729 | 0.736 | 0.013 | 0.022 | 0.031 | 0.042 | 0.256 |
| | mRNA-nucleus export | 0.419 | 0.706 | 0.039 | 0.018 | 0.268 | 0.016 | 0.294 |
| | negative regulation of biological | 0.187 | 0.161 | 0.093 | 0.122 | 0.033 | 0.036 | 0.005 |
| | process | | | | | | | |
| | phosphoenolpyruvate-dependent | 0.558 | 0.567 | 0.033 | 0.036 | 0.564 | 0.025 | 0.557 |
| | sugar phosphotransferase system | | | | | | | |
| | translation | 0.344 | 0.55 | 0.374 | 0.033 | 0.011 | 0.063 | 0.007 |
| | nucleotide-sugar metabolism | 0.781 | 1 | 0.038 | 0.031 | 0.033 | 0.06 | 0.125 |
| | cation homeostasis | 0.52 | 0.742 | 0.008 | 0.001 | 0.416 | 0.036 | 0.04 |
| | di-\, tri-valent inorganic cation | 0.362 | 1 | 0.013 | 0.008 | 0.454 | 0.049 | 0.071 |
| | homeostasis | | | | | | | |
| | calcium ion homeostasis | 0.168 | 1 | 0.004 | 0 | 0.341 | 0.036 | 0.022 |
| | negative regulation of cell | 0.033 | 0.097 | 0.424 | 0.136 | 0.003 | 0.011 | 0.083 |
| | proliferation | | | | | | | |
| | cell homeostasis | 0.582 | 0.645 | 0.003 | 0 | 0.179 | 0.037 | 0.018 |
| | signal transduction | 0.893 | 0.978 | 0.007 | 0.203 | 0.008 | 0.025 | 0.005 |
| | cell ion homeostasis | 0.541 | 0.807 | 0.008 | 0 | 0.4 | 0.041 | 0.037 |
| | metal ion homeostasis | 0.258 | 0.914 | 0.009 | 0.001 | 0.54 | 0.021 | 0.059 |
| | viral life cycle | 0.364 | 0.849 | 0.092 | 0.008 | 0.057 | 0.056 | 0.032 |
| | tRNA modification | 0.208 | 0.359 | 0.035 | 0.004 | 0.001 | 0.053 | 0.017 |
| | amino acid activation | 0.473 | 0.627 | 0.167 | 0.022 | 0.009 | 0.147 | 0.097 |
| | regulation of cell shape | 0.651 | 0.661 | 0.003 | 0.049 | 0.333 | 0.034 | 0.065 |
| | excretion | 0.723 | 0.038 | 0.053 | 0.029 | 0.365 | 0.033 | 0.093 |
| | traversing start control point of | 0.66 | 1 | 0.003 | 0.004 | 0 | 0.037 | 0.053 |
| | mitotic cell cycle | | | | | | | |
| | histidine catabolism | 0.603 | 0.584 | 0.536 | 0.562 | 0.554 | 0.028 | 0.042 |
| | NADP metabolism | 0.739 | 1 | 0.065 | 0.012 | 0.009 | 0.143 | 0.057 |
| | histidine family amino acid | 0.603 | 0.584 | 0.536 | 0.562 | 0.554 | 0.028 | 0.042 |
| | catabolism | | | | | | | |
| | protein-nucleus import | 0.902 | 0.779 | 0.062 | 0.016 | 0.258 | 0.025 | 0.031 |
| | purine base metabolism | 1 | 0.369 | 0.068 | 0.049 | 0.07 | 0.032 | 0.05 |
| | negative regulation of cell cycle | 0.036 | 0.011 | 0.097 | 0.332 | 0.1 | 0.011 | 0.011 |
| | response to DNA damage stimulus | 0.172 | 0.466 | 0.197 | 0.449 | 0.128 | 0.021 | 0.013 |
| | tRNA aminoacylation | 0.473 | 0.627 | 0.167 | 0.022 | 0.009 | 0.147 | 0.097 |
| | negative regulation of protein | 0.439 | 0.874 | 0.311 | 0.041 | 0.202 | 0.065 | 0.016 |
| | metabolism | | | | | | | |
| | sphingolipid biosynthesis | 1 | 0.536 | 0.089 | 0.107 | 0.332 | 0.015 | 0.039 |
| | response to endogenous stimulus | 0.181 | 0.7 | 0.391 | 0.42 | 0.157 | 0.02 | 0.042 |
| | tRNA aminoacylation for protein | 0.473 | 0.627 | 0.167 | 0.022 | 0.009 | 0.147 | 0.097 |
| | translation | | | | | | | |
| | regulation of angiogenesis | 1 | 0.788 | 0.766 | 0.13 | 0.001 | 0.529 | 0.036 |
| | nuclear import | 0.902 | 0.779 | 0.062 | 0.016 | 0.258 | 0.025 | 0.031 |
| | phosphoinositide biosynthesis | 0.768 | 0.552 | 0.189 | 0.036 | 0.202 | 0.029 | 0.216 |
| | DNA repair | 0.241 | 0.721 | 0.172 | 0.219 | 0.106 | 0.008 | 0.012 |
| | intracellular transport | 0.114 | 0.143 | 0.001 | 0.056 | 0.156 | 0.003 | 0.027 |
| | cell surface receptor linked signal | 0.3 | 0.368 | 0 | 0.102 | 0.031 | 0.132 | 0.005 |
| | transduction | | | | | | | |
| | vasodilation | 1 | 0.277 | 0.558 | 0.569 | 0.046 | 0.254 | 0.03 |
| | cytoskeleton organization and | 0.178 | 0.149 | 0.006 | 0.044 | 0.169 | 0.009 | 0.126 |
| | biogenesis | | | | | | | |
| | monovalent inorganic cation | 0.11 | 0.147 | 0 | 0.017 | 0.001 | 0.002 | 0.004 |
| | transport | | | | | | | |
| | complement activation\, classical | 0.828 | 0.261 | 0.182 | 0.173 | 0.034 | 0.34 | 0.021 |
| | pathway | | | | | | | |
| | glycosphingolipid metabolism | 1 | 0.307 | 0.095 | 0.125 | 0.096 | 0.003 | 0.043 |
| | protein targeting | 0.106 | 0.786 | 0.001 | 0.031 | 0.019 | 0.002 | 0.011 |
| | humoral immune response | 0.072 | 0.564 | 0.276 | 0.15 | 0.029 | 0.157 | 0.026 |
| | regulation of cell proliferation | 0.163 | 0.125 | 0.491 | 0.206 | 0.014 | 0.006 | 0.059 |
| | regulation of vasodilation | 1 | 0.277 | 0.558 | 0.569 | 0.046 | 0.254 | 0.03 |
| | humoral defense mechanism (sensu | 0.259 | 0.575 | 0.311 | 0.097 | 0.012 | 0.307 | 0.025 |
| | Vertebrata) | | | | | | | |
| | mitotic cell cycle | 0.211 | 0.244 | 0.095 | 0.046 | 0.036 | 0.006 | 0.046 |
| | translational elongation | 0.461 | 0.326 | 1 | 0.601 | 0.05 | 0.054 | 0.013 |
| | activation of MAPKK activity | 0.07 | 0.541 | 0.54 | 0.549 | 0.038 | 0.228 | 0.035 |

*Based on the version of Hs-Std_20050713 GenMapp gene database

What is claimed is:

1. A method of diagnosing nasopharyngeal carcinoma in a subject, comprising:
obtaining a nasal sample from the subject,
examining in the nasal sample an expression level of a gene involved in the Gα₁₂ signaling pathway, and
determining whether the subject has nasopharyngeal carcinoma based on the expression level of the gene, wherein an increased or decreased expression level of the gene relative to that in a nasal sample from a healthy subject indicates that the subject has nasopharyngeal carcinoma.

2. The method of claim 1, wherein the gene involved in the Gα₁₂ signaling pathway is selected from the group consisting of Gα₁₂, Rho guanine nucleotide exchange factor 12, RhoA, SLC9A1, Rho-associated coiled-coil containing protein kinase (ROCK1), profilin 1 (PFN1), and JNK, and wherein an increased expression level of the gene relative to that in a nasal sample from a healthy subject indicates that the subject has nasopharyngeal carcinoma.

3. The method of claim 2, wherein the gene involved in the Gα₁₂ signaling pathway is the Gα₁₂ gene.

4. The method of claim 2, wherein the expression level of the gene is examined by determining a level of the protein encoded by the gene.

5. The method of claim 2, wherein the expression level of the gene is examined by determining a level of the mRNA transcribed from the gene.

6. The method of claim 3, wherein the expression level of the Gα₁₂ gene is examined by determining a level of the Gα₁₂ protein.

7. The method of claim 3, wherein the expression level of the Gα₁₂ gene is examined by determining a level of the Gα₁₂ mRNA.

8. A method of inhibiting nasopharyngeal carcinoma invasion in a subject, comprising administering to a subject suffering from nasopharyngeal carcinoma an effective amount of an agent that suppresses the Gα₁₂ signaling pathway.

9. The method of claim 8, wherein the agent is selected from the group consisting of
(i) a small molecule that inhibits activity of a protein involved in the Gα₁₂ signaling pathway,
(ii) an antibody that binds to a protein involved in the Gα₁₂ signaling pathway, Gα₁₂ and inhibits its activity, and
(iii) a compound that inhibits expression of a gene involved in the Gα₁₂ signaling pathway.

10. The method of claim 9, wherein the protein involved in the Gα₁₂ signaling pathway is Gα₁₂, Rho guanine nucleotide exchange factor 12, RhoA, SLC9A1, Rho-associated coiled-coil containing protein kinase, profiling 1, or JNK.

11. The method of claim 9, wherein the gene involved in the Gα₁₂ signaling pathway is Gα₁₂ gene, Rho guanine nucleotide exchange factor 12 gene, RhoA gene, SLC9A1 gene, Rho-associated coiled-coil containing protein kinase gene, profiling 1 gene, or JNK gene.

12. The method of claim 8, wherein the agent is one or more small interfering RNAs (siRNAs) that suppress expression of the Gα₁₂ gene.

13. The method of claim 12, wherein the agent is one or more siRNAs each containing the nucleotide sequence selected from the group consisting of:
(1) 5′-GGGAGUCGGUGAAGUACUUUU-3′,
(2) 5′-GGAUCGGCCAGCUGAAUUAUU-3′,
(3) 5′-GGAAAGCCACCAAGGGAAUUU-3′,
and
(4) 5′-GAGAUAAGCUUGGCAUUCCUU-3′

14. A method of screening for a compound capable of suppressing nasopharyngeal carcinoma invasion, comprising:
contacting a candidate compound with a nasopharyngeal carcinoma cell,
examining a level of the Gα₁₂ signaling pathway activation in the presence of the candidate compound and a level of the Gα₁₂ signaling pathway activation in the absence of the candidate compound, and
determining whether the candidate compound is capable of suppressing nasopharyngeal carcinoma invasion, wherein the level of Gα₁₂ signaling pathway activation in the presence of the compound being lower than that in the absence of the compound indicates that the compound is capable of suppressing nasopharyngeal carcinoma invasion.

15. The method of claim 14, wherein the level of the Gα₁₂ signaling pathway activation in the nasopharyngeal carcinoma cell is examined by determining the expression level of the Gα₁₂ gene in that carcinoma cell.

16. The method of claim 15, wherein the expression level of the Gα₁₂ gene is determined by examining the level of the Gα₁₂ protein.

17. The method of claim 15, wherein the expression level of the Gα₁₂ gene is determined by examining the level of the Gα₁₂ mRNA.

18. The method of claim 15, wherein the level of the Gα₁₂ signaling pathway activation in the nasopharyngeal carcinoma cell is examined by determining the expression level of the IQ motif-containing GTPase activating protein 1 gene.

19. A method of inhibiting nasopharyngeal carcinoma invasion in a subject, comprising administering to a subject suffering from nasopharyngeal carcinoma an effective amount of an agent that reduces the level of IQ motif-containing GTPase activating protein 1 (IQGAP1), wherein the agent is an antibody specific to IQGAP1 or an interfering RNA that suppresses expression of IQGAP1.

20. The method of claim 19, wherein the agent is one or more small interfering RNAs (siRNAs).

21. The method of claim 21, wherein the one or more siRNAs each contain the nucleotide sequence of
5′-GAACGUGGCUUAUGAGUACUU-3′,
5′-GCAGGUGGAUUACUAUAAAUU-3′,
5′-CGAACCAUCUUACUGAAUAUU-3′,
or
5′-CAAUUGAGCAGUUCAGUUAUU-3′
